SEQUENCE LISTING (4 sequences)

SEQ ID NO: 1 - DNA, 20885 bp, Homo sapiens
gctagtgcggagttttattggctacaaaatagatgcaaaatgatgagaatctgaaggctgcagtaggaaagtagagctttaccctcataaactcgcactttgattagaaaagtgcaatatattaagagcattatgagaagtctggtgagactgttacagaaaaaaaaaataaaagtttctgagtctgataattccaagggtatcttttagaactcactcactggtgtctgtgcaaggactttccttgggggaaaatagattttacaacaggcggaaactttcattggtctcatgcgtgcttttggatttcattcacttgacaaagaactaatcttccgttgatggtctcctgggttatggccttgatctttggagttgcagacactgagaaaaagagcatggaagatgtcactgccatgcttcttccaccatctgaactgtactcattttcatttctctgctgtcagcccctgaaggctctcagtgccttccaaatgatcactaagcaatgaggtgggctctgctgctgggaaaagggatttcccaaaggagggattttctcccactctccaggaaatgaggatctctgaacagtgtcaccttctggaggtgaagaccttcatcataggtgtgcctcatatggaacctagtctctgaccacaagcacttgaggagttctttctctgcctgtgggctttgtagcactgcacagattccttgagcttacagagcacttagtgctattcagggtacctcattttaaagtctagaaagcatgccacatgaggaatgggtgagaagccaaggatgcttaccatggctgttttcaaatatataacttatttgaataagtgcctgttgaaacatcaataaagcaattctagaccatagggaagaattgaaatcattaataaatttacaagggagaattttgttaaattaaaacctctccattgggaccatcccatcaagtatgggttccccatcctaagacatgtgtgaagagagtctcaaagacccttgtcttattttgtagatgggtagcagtgagaccagctgtcctgtgatgattactccaactgattctttgaatctccaatgccttagaataggtttatcacaaactaatcagaaagtgccacttgtcccagactacagtctcctgcaacttttctgtgagatattaaaggtaatctgagaaagcatgtttccaggtcaaagatgtttgggaatactgggctaaacaaagttaaacttttgttgtttctacctctttcttgttccttttctccttcaaagcagtacctgttccatctttaatacactgaactgcaacaggactgtccaagaagaggatcatggcacacagtatttctaaccttatctggccatagaacacagtgttcgtaatacactattcaaatctcatggaacacgagtgttcaacagaatcatctgagaaatgaaatcatgaaatcattgcctcttagttttctaggggtttagtaaactgactccattacttaatagagacataatactattaattatcattttaattttctttactgcttgcttttacctatattgaggacaataatgagagcttacatcattttgtattagctaatttgttcattgaagattcctgacccaggacattttgaacccaggacatttgggagtacattaaggattatgactactacaaaatggataaccatgctaattccttaaaaacagagattcaaacaatgaaggaaattttttacctttcatgctcgactttgattcttcccgtgtcctataaaagtgataaaggttaaagctcactttagtattagcaacaatcataacaacttctcaggatgaactaacttccttcgcttgcttgtaaatcctctgctctttaatgaggataattgtctgagcttagtagtctcaaggtcatgggttcacgggagaacccgtctggatacagaaggcattgggtaccgcctcctgctaaccctggagtcaggggaagtccaccaaccatcccctcctttcagctgcaagcttggttttctctaggatccctgccagggtctgggtctcaagcctgagacccctgagcttctcaggctgttcaaagtttggtctagtgatgttttatttatgtatttttataaaacttctatcccttcctttcggatcatagaatatatatgccctttatatggccagctgttgttatttcctggggtggccagaaatatcagattgaaagtctagacatctctagctcatatgcatctttttttttttttctcttttgctggtggactaacacattccccctgccttcttttttttggagccaaaattgtgtgcattcctactgggaaacacagtggccaaatccttttgaattgtttccttctagagactttaactcttctgactgcaaatcttagtgtcctgtgagtattagttgattaattatacttgctgcttagtgaaatacagccagctataggtatcttctggagtagctcaacacaacttttctcttgctagagtgactcttgctaacagaacccaaagatgcacacatatacccacaggagctggaggtccctcgcatgctcctctcgtgccagcctttgccttacccttcactctctccctccaggagccgtcggtacgtggtgatttccttctccaggtgggttttgatgcccagcagcacttggtattcattgttctgccgctccagttcatggcgtagctgcgtcagttcctcctcatagtgggagatgatctcttgcatgtcctggagcttgcaggagtaccgagactgggtctcggataacatgttttccaaagcagatttctgaaagaggaaatgatcttctgttaattaactgatggcttgattgagtcaataacaggtgattgttgagtacttagcagatgtcgaccagttggctaggtgtcttggtagctgctgaggacaccaagatgagaagtaggctgacttacaggtttgggacttgggcagtcaggagttgagcagtgttaggacttttgaagtccattgccttgctctgaactgttgtttaactcctttcattttatctttgacctgattgaaaactcccaaagagcgacagcaactgggactgctatctctttgtatcttcggagaagggtctcaattagggatgccagataaaatagagaacatctagttaaatttgaatttcagatgagtaatgagtaattttttagtgtaactatgtccaaaatattgctccagtattgcatgggacatgcaaaaaaaattgctttttatttgaaattccaactaaactgagtattctatattttaatttggtaaatctggcaaccctagtctcaagcaatgctttttgattatgacgatggtaaaaaaaaaaaatgtcattttttgaggaaattctaacagctgccaaatgaagtttctgggctagaatatggaggattgagttcaagtcccagttctgtttgcatgtaacacctgcttttctgaagcgcataattttaaccttaaagagaatctcctaactgtgctggatactctccctttatcgtggctgctaattttcagggggccgttcatgctgcctggcggttaaagcacatttccctggtcagccagtctcctactctcccaggaggttattttgttccctctcctctgtccttgccatccttcctcggagtccatggcctcgcttcctaattttgttgatgaaatgaaagcaatcgggagagaccgcccactgctccctacattgctcccaggcacatacttgaatctgcatctgggtgctggcttccctctgttcttttgtgtgagctgtctgtgctccagtcatctcggcagttgcccgccttcttgcgttatcaatattctctctggattattcccaccagctttcaaatagaataatttttcctaccttaaaatatatctttcttaaacccatatctaccttcagctatcatccaatttctctgctctccttcagctacagcaaagctcctcaaagagttgtctatgcctgctgttcctcttgagacccctccaaggtagcctacaaagccctacactgtctggttctgttgggactccgcggcctcaccctgtcttcctccctcttcccctcccttcccctcctcttctctcctttcttcttcccttcattcactcactgtagccacatggacttccctgctgctcctccaactcatcagacacacttgtgccttgtcgcttttgcacttgctgttccctcctcttggaacacatcccatcaccacatcaagcacctccttacctctttgctcactgccactctaacaggaaaacctttcctgaccaccttgtttaagatagcagctcttcaccctggtgcatctgccctcctcctctgcttcatctctgtagcaattttctccatctaacatatattttgatgatttgcacatttattatctgtcactcctactagaatataaactccttcattaaaaaatcctcagtgctaaggattggtgtcagatagtagaatgcatgaaaatgggtatacatgcctcgcctgtttcacagggccattgtgaaatactgtatgttaaaacgttttgaaagttgcactataattatttttgtactcatgatctctacgtgcacatttgcagtgtctgttcaccatcacggatgcataaaacacagcagtcatttacccaagtgcacctgcaacataaatgaacaaatcattgcctctcaagactgagggtagaatcgcacttggcaattatatggatgaatgattagaagactcaatagggtatactgggaatcaaatgatagaccttcataaattttgaatgcttatgaccatgctaagcatttttctaaactgtgaagatgatagaaatgacttggacacaagaacttagagttttctgggtgataagacatatacataaatgaatagggcagtgctttggagttagccaggcctggatctgccacctaccatgttggcactggaatttctctacattggggtttgaggggggtagcctggttgtaagtggagagggcactgatagaaattatgggggtctagaatcccccttcctccaaataaatcatatggtttcatcaccagttacttactagctgtgtgaccttggccaatttattttactgttggagttttggtttcctcatctctaaaatggggacactaagacctatctgggaaggttactgtaaggattaatgacatttaatatcatgaactacttagcacaatgcctggcaccatcgggacactcaatacatggtagccctttattatggttctcatcataaacaatttggatggaaagtggaaagtgagagttgtcccaagggagttgagggtcagggcctgggagagaggtctcagaaggcttcgtgaaggaggtggtgtgggattcacggttttcctagccttgactcaccgtgctgtactgtgtctgcaggtcaatctccagggcctggaatgtgcgcttcagttcgtggatgtcaccttgtctgctctgcacagtggctggactggctgcctcctgggacatggctgcagactgtgggaccaagcaaggcaaaggcgtcagcattgggacctcatggaaatgcaacctttgggaagtttgtgcatctttcttttacctgttctttataccaagtgtccaagtctcgatgcttcttctttattataagctcatattcttgtctcatatcctccaggaccttaatcagatcttccctgggacctgtatccaccttcacattgacattgaagtcacttggcacatgatgcttctccatttcctatttaataacagagaaaggaaatgaaccggctttgacaatcagaaggctggattgccatgttgattggcagtggtaaatagatcttttaaacattccgattatgtggttgctcccactgtcactggtttggtttctcaaatattacttacttgttggtgataaaacattgtatacttttttctctaataattttataaaataaagaatatctttgagaccaacctgaccagcatggagaaaccctgtctctactaaaaatacaaaaaattaactgggtgtggttgtacacacctttaatcccagctacttgggaggctgaggcaggagaatcgcttgaacctgcgaggtggaggctgcagtgagccgagattgcgccactgcactccagcctgggtgacagagtgagaccctgtctccaaaaaaaaaaaaaaaaaaaaaaagaatatcataaggcaggtaatcaaatagcctccatgattactgacttgatccgtggttgaaaatatttcagaaagaattatgcttccatagtgtgcaaagtcctgttcagcagatgtggattggtgaagtgacaacatagttaagcagctgaaccttacaggctataggccaaagcttttgggaagcagttcaagaggcaaacagcagaaagaaggtgtagaggctttcaaaatctcatcaatgccttgtgacactgtagggctgacgcccataggatttcatgaaaaccaagcagccacaggaaaggtttaattaacaatcattgtctaaccagctattcagaaactgagctctttgatgttgagaagcaattttgttttcctaaaatgggaactgtttttctatcattttatcagagtcttgaggacttgtgatggatggtaactctaaaagtcagttaaatgcttctggaaattaggattctttatttttgccccacatcacacttatcttggttggtagcagactctcttcattatattgtcccctaacttgctgaacattgggccagtgatggaagtccagtctcacgcattgattcttatagtaacattaacaagcacatctaggtaaacattgtgtattaacaaaactgttgaccaattcatcattaattatgattgcattttggttgttgtatttgacatgtgactagctaaccttgaaattagaaacattagatatttgtatgtttttctaaagactatttagggtgaaggacaaggggattggcatccaggtggtggtttcttctgtagctgtcgaatcagcctgagatgtgcaggctaagagctgtgcttattagcaggtgttcctggggagttgtacctgctcatggtgcttcttcatgagaatgagctctttcctcattccttccacctcctgttctaggtctgttgtgacaatggtcaggttgtctaaggtccttcggaggccctcgacttcaatttccaagtctttcttaaaggagtgttcattttcatacctttggtgagaaggaagagaagagtgcactcattgttggaaggccatggtttttgaaaggattcattttcatttagaggtgaatcttttactttttaggtttaaagagagcaaatgaatacatataggatgtcttttttatgccagtttcatgctacacttctccatgatggtgactcaatgtcaacttttaaattgagtagggagaataggataataaacagtcatatacccattatctatatttagcaaccatgaatgttttgccatatttgcttcatctatttcattgctgaagtattttaaagcagattatagatgtgatgacattttaagactaaatacttcaggatgcatcttttaagaaaaatatattttaaaataaacaggccgggcacggtggctcacacctgtaatcccagtactttgggaggctgaggcgggcggatcacgaggccaagagattgagaccatcctggccaacatggtgaaaccctgttgctactaaaaatgcaaaaattagctgggcatggtggtgtgcacctatagtcccagctacttgggaggctgaggcaggagaatcgcttgaacctggaaggcagaggttgcagtgagccgagatcacgccactacactacagcctggcaacagagcaagactccgtctcaaaaataaacaaataaataaataaataaataaataaataaaataaacataacactacacctaaacattaataacaaccctttaatatcatctaattcttgattcatattcaaacttccccatactataatgtgctttatatctgatttgtttaaactaaggttcattcaagtaacacacagtgcatttgccgttgctatgccttttcattataagattttagatgcgggtaatggcaagatgagttttcctttttttttttttttttgagatggagtctcactctgttgccaggctggagtgttagtggcgtgatcttggctcactgcaacctccacctcctgggttcaagtgattctcctgcctcagcctcccgagtagctgggactacaggcgtgtgccaccatgcccagctaatttttgtatttttagtagagatggggtttcaccatgttggccaggatggtctcgatctcttgacctcgtgatccgcccgccttggcctcccaaagtgctgggattacaggtgtgagccaccgcgccccgccgagttttccttttgtaaagcaaacaaacaaaaacccgaacaaccctgtgaagaaggaacttacttgaggttgaagtcatccactgccatcctggcattgtcaatgagaagaataatctgagcattggtcatcttaccatccactatctgtaaaacatgcacaaaagcacaaaaggttatctcacttggacacacccacacatgaccgctgcatgtctgtcctctagcttcctgaatatttgacttgttagtacagacaacttcttgttctggcagtggaacaatgaataaccgaatcagtaaatgcttagtgagcacatactaggtgcaaagcagaatgattttaaaataagaatatgatctgcagtcagtgttactgccttttttctttttatgttgagttttgacaggcttggattgatgcaactgctgaaagaatgatataggaaggggaaggacagtaattgttcaaaatgccctctaactttttaggttatggacatgtcatagcattttgcttaattaagctcatccacagctgaagatgttaaaaggactttaggttctgatccttttcttattgtattaggttggtgaaaaagtgatttttgccattacttttaattagttcagccctccaaaaatacaaaaattattatttcaggtgaacataattcttacttgtgatagcaatcatgtttctaatcagtgttactgagcaacgctaacactaatattcaatgtattttgtaatagtcatcacagaacacattttttttttgcccgaagtgactgtaacttgaaggtgtgtttatgtcattgcaacttattctgtgatgagaatgtgctcattgtagctattaaaatattaactgaaataattccttaccatctaaggaaaatgcttaagaagcttcagtagtggtaaagcctccagttaaagactttcactagatgttcgtataattttttttttactttgaaaagaaaaatagtattgaaagcacttactcgagcaagtgtgtatacatttatgatttgtaaacacttgtaggtatgttatagtcagagaaatagcacacatttgatgtatgtcactgtctttagggtagaagatgtctcgttgtcaaaagtgtaataatgcttttacgccaagtgttgtgctggactagaaacttagagcttttctttggttctggctctgttcttgaccatccatgtggatgcatcaggctgcatcactctactctccaggcctcagtttcatccattgtcaaataaggagtctgtactagagacagaggaccttagaggtcttagctctaagtttctgtaatttagttggagtcagagaatcctgggtaattcccattatatttacatagctcattcctttatttagaaagcattttcttaggacaaagctatacttcagtacaaaggcaccttttaaaagtctggtcctatttaaagctccactgatcccatggaatttggtatcatgaatgcacaatagcaggatttagagatggcatgaatgctgggtctggaactcaaagagcagggcctgggatcctgctccgcgcttactggctgggtgagattggctgagtgagcttggctgagtctcagtttattcatccataaaatagcaataacaatagttacttcatcaaagagttatgagaattagaagagagtatgtcaaataatgtcaaatagcatattatcggcatattatagacagtgaaaaattggtcgaaactgaatttgttcagaaaaaagtaaataatacacctttctttttaaaaatctagcacttttatagattcagtgactacactgtgaacatccccaggttctatatgctgagatgcctgctcagacttttgctgttcatggtgggacatagccaggctggctgctcctgcctcattctttttcctcttgtgggattccaaatggtactatggccccttcccttgctataaagaaggacacttttagatatttccttgtctcctctccacagtgctgcttaggggcattcttaccaatacttctccctccagaccccttattctttgcattatgtaagctcctgcaaaatgaaaagtagctctatcccaaagagcctgggggccattgtggggtaacccttacttacttgtgcactgaggttttctatttgacacacactatgaggtgttaatctacatccagagaatcagaaaatcaaaaatactgtctatgggcttatttagcttttctgggttaaattggtaatttatccattgcaaagtccacctcaagtgtctgattcccccagctatcattctcatagcacacaaatgtgaattctgaaatgtgcttgatatagagcacgtttaaaactatttcccattattcaggcttacttagtacagaattatgatataaccatcatttctgacacaatcttcatggtaataagtggatgaagatgggttgagaaacgatatatgaggcataggttttttactgagtgttgtaattaatcacccagattctcctgaaggccctcagttcattcttttaaaagcaagttggcatattcttgcagcattgttgcttcaggatgtttgatctactagggagaggaaaacagaatgtttgactgtaaacaaaaatgccaaatatagtccgattgagaatcattcaaggtgttttatttatgatactcctatgactgtgtccaattctggatttcaaaaggattgaattaatttttaagctcaacttgttatcaaccttttgagtgatatttaatatacctaaagtgagggtacgggagaactgtatccactcagagcatttcaccaaggtccaggaaggataagccccaggtaattgtgggacctccttgcacctctgttttctcatcttcaaatgagggaggttggggtagatagacaacctcaactttaaagagtttctttcttcaagtgtagagaaaacacaatgttagaggtaaaaatccagaaaagagtgaaattcaaagccaaagaagtttcctattcaatctcagaacccagaattccaggtcatctgaagggtgggcaggttgctccccacaccccccattgcacctactcacttttctcaattctgttccaaaggatttagacaatgattaggtctcagtctccttatctgtaagttgagaatacaaatacctgctccacacagttgtgaggattaaatgagacaatgtgtatgaagtatgtgatgtgtggttaatgatgcaagtataatgcattatctttattcatactaagtgtcatttttatatgaggtgggctaggcagcactacactgtagtgaaatttatgaggctactggaaacagacctgagttagaatcccacctctgcttcttgctgatggctttggatggctatttcacttttttccaagcttccatttccttacagggttcttgcagaatcaaataaaataatatggaacatttccatcattgcagaaaactcttggatagtgttgacatgtgtattaaatatccagcaggatgcctaggtacataataagtgctcagtaaacatttccctctctttcctccctttcttttttttaaatctcttccgcgagctcataaattatttgactggttctaaccttgtatttcttttgggagtcatggtctccctttgctctccatttgcaaaaatcttcaattcctcctttctacaaccaaagcaactttattaaacaaaaaaacatgatgaggactgttgagtcaccttgtttttttttttttttcataaagacaaccatgcactatatagttaaggtaattacagtcagttattaggttgattattcaataattagggttcattacaaggacagatttatattatcttgactggtccttcctacattcaagcaaggatgactgagcattgtgaaataatatatataaatattggaaacttgtgataccctttctaggggaggttaaatcatggtcttacagggaattttctgggactgctttatataagaggggagttagatagctttaaaacataacctatgagatgattggattccttctacttctagctaatgttatgagacatcccagaaggtatttgcttagcttttttgaggtttggttaaagagttcggttttttttgaacaaggtctttatagccatctcaactctcagattctaaaaattatgctcattttattattattggttctagcaatatgccattgtcagaactgtgagcaactttactgcaagcaacccacaaacggaaatatctagggatgtcatgatgacctatatgctggtgtggtgacaacttttaggcataatcttcaaaatagtgtaacagccttttgtcgatggataaccacattgttcaaaaaagtttgaggtggagcagaactgaggtaatgagaaagtaaacactctcttatgcctggtgcactgagcatgcagcatgaacaccagcccttatcatttctatgacatcgttgggtcataagtgacattaaacagcagctggttgcctcacattttaaagatgtttttggtctgtgagaaatcagtagtctcatttttgaaacagtctgtagcacttctatcatttttgctgccttctttttctacattatccatggacagtagactaattaaacaaaacagacgtttcccactccaaatttaagatataataagaaaacatcccccaaaacctttgtgcacaattacttcaagcgctgatgccattttcctggaaattgtcttctggactcatttttcctcccgagggtcccctggcaaagcagccccatctggatctccagggtcacccagcatcttacctgctcctgcaggtgtgtgatgttttcctcatactgggaataatctttcttactgccaggatctctctgctggtgccatttcaggatgcggctttccagcttcatgttggcctcctccagggcgcgaaccttctccaggtaggaggccaggcggtcgttgagattctgcatggtggccttcccatttccgcctagtagggggctgcttcttccagaaccccaagaccctccagggggtgggcagctccgcgtggtgaaggacagggagatgcgggctccccccgcaccgccatggacggtgggagccctggggaagctcctgggccggccccagccacctccggcgccatggaaggaggccgagggggtctggctgaagctgtgtccggagttcatggtcccatctgtgtttgggacggggctgagctctgctccactccctggcaccgcagaactgagccgccccagactgccctggatggttttatggcctttgctgtgggagttcccttctctgacaattgtaccaacaagattgtatcattgtgcaacttgtgtttcctcttggtcaatcccaaagtgcccctgggccttcgcacactgttgttgtttagagccacatcaatatgacagtttgcttcctcccttcccctggtaacccggaggtgtggcccacgacatcaggcgggtattgggctcaggtatctttctaatcttgccgtgaagtttttccattgttcatttacctggaatgggttaggttaaagtccacccaaaagaaagcatattcgatgtagtatagatttccttaaaaaaaaccaaaaaccaaaaccaaaacaaaacaacaacaacagcaacaaaaaaccagacattaaacaacagagatacacacacctttttccacccccaataagccccacctctaaagggagggtgagcgagcctggagaagggaaagccagctcagaacaggatctcaggtcccagaataaagcttgctgtgctgaggcagccgagaaggtggtgaatatctgatgcctttgtggcagtcactgtttgaaggaaagtactttcaaaggactcgctcttaagaacaaaccctcaattatagatgtaattaattaatgaatgaccgaaccttccacttccagggtgcctttcatcccagcacacctcaaaaccaaacaaccctggcctgcgctcaggatttcccttggccctgggggaggaatgtagccgttgttagcagccccggaccccggctgagcctgccacggctgggctctgtgtgtctgtgacctgcagagggacgatggaaaacaggaaacctgccatgggctgtcaaatgtggttgatgaatcttatatttcatctgccaaggtaaaggttaggaatgaataaagaagcagtctgggggcgggagccacgtgctagttactggcagttggatttaaaggggacttacaaataaaatatcagcgcaggaagggcttctggcctttctggttccaagccactaattggacagatgaggaaagctgaggcctatggtggtggggaggttagtggcagagccagggctagattctttctccacatagcactgttatcttgggttgaatcttgacaggtcctgtaccacacttctctttcataataataggacttgccaattggtgtagctcttttatattctttattgaatcctcacaactctttatgtatccaggcaagggttgttgtcttcattttgcagacaaagaatcagggtctcatagggatgaatgatccagagctgattcgcatgtacagtgtaattgagcacatgaattcataggtgacactgcccgagtctgtgctgtcctccctgcctggaaagtcactcacccctacctcttctatagttcaagggctacctccttcaacaatcattttctgaattctcctgcatgagttgggtgaccctctaggaggaaactgcctcttctggatgccgaggtcctccagaggggcagacaccttggcatcccacctcatggtggaagctgggagcccctagccaggcgcagccacctcctgtgcctgggatgaagtccactccgttctttcactccattatactgcatttgccacacccttctgagaccgcaggcttcttgaaagcagagtttatgtcttccatttttgagcaccaaagtctggtttcttggtggtattttgcaaatgtatgttgactcaatcataaactttttagagtcaggattttaacacaggtctcctgatgccatcaacttctgctccttttaggactgtgagttatagaactagaaagtaggaaaacgcctctcctgggtatagattaaccatccatgaaggccaatcttttccaattacaaataaagccacccaaaagcacaccagctctgctcctcccagtcttacaacaaaaccttatcagggaagagtctctgaatattttttatttcactgtgcacgagctggaaaaatgagcctccaagcctcagggaagcttggagagtgcttagaaagagtgccgacagtcacttacatctcagatatgagaaaaagacaaaagagacaagtgataaaaatagagcaaatgagggaactagagagacttgtttggcgagtggtggcaaccagtgttctgtggatgtggacggttcctggtcatcaaagttatggtcagctctggttcctgttcgcctggacccagacttaccaaggaagaaaacatttacctttagcatgtgcaagggccgggctgcactgcctttttctaaattctctcaccagaatctacaatgaaattaatgcttctctaattatttcaggcatgtctgagtggcagtgacttatcaaccccatagatttggccactgtctcattttatgtccatgtgtgggagttttgatttataggacttatccttgagcaaaagatttcttgtgtcatagcaggaccagggccaggaaaaactggtggtgagatctgagagcactggggagaaatagtctcttctagtactgttatatgatggtttcccataacttctttttttttttttctcaagatggagttttgctctgtcacccaggctggagtgcaatggcgcaatcttgactcactgcaacctccgcctcctgggttcaagcgattctcctgcctcagcctcccgagtagctaggattacaggtgcgcaccaccatgcccagctaattttttgtatttttagtagagacggggtttcaccatgttggccaggctggtcttgaactcctgacctcatgatctgcctgccttggcctcccaaagtgctgggattacaggcatgagccaccctgcccagccaattttccataacttcttttttttttttttgagacagagtctcgccctgtcgcccaggctggagtgcagtgatgtgatcgtggctcactgcaacctccacctcccgggttcaagcgattcttctgcctcagcctcccgagtagccgggaccacaggtgcgtgccaccacgcccggctaatctttgtagtttttagtagagacggggttttaccatgttggccaggcagatcttgaacttctgactttgtgatccacccacctcggcctcccaaagtgctgggattataggcatgagccaccgcacccggccctggttttccataacttctaatgtgtagagttccattggattttaaaagaaatgatgcttttttaaaagcatcattttaaaagcattaaaagaaatgatacatgcttattatacaaaatcaagtgatacatgatggtacaggaaaaaatatttaaaaattctaccacacacacacaacgtttggtgacatttggtgaacatcctttcagatactatgtgtatgtattggaagacaaaagaatggctagaataattggaaaaatgggctcctgcgacttattccattttagaaaaacttaagtcaattaacacattttaattggcaaataaggaactaaaatgaattcactttagtgacatagtttttctagaatttaaaaacgttcatcacattcttttaaattttacatttttaagaaggataaaatggcctgtaaatgccaggatttttggaatcgcaggactggaaagaaaagcaacgctgagccacttgttgcagatttttgcttttagggaggacaacggcagagtgctgagcttgtttttgaagtattcttaaagaacgcgagggatgaagcctccaactgcttcctcagtaaatggccaacatttactgatccttttggttcacaagcatttcacttgcaatatggtttcaggatttcttttggtatttctgtagagtgcaaattgttcagaatcaaaatcattctgctttgcatgattttctggctgaaacatttgtagttttcacatttgattcatctttctgatgaactcagttgtcgtcttgaaagggagtgggtactttgtaagacagctcagctgttgagtgaatgcatggggttgcatgtgggtgtgttggggtcagaagggagccctgtcagaaggtagctgtcatgaactgggtacaccgtctgtttgtgtgccttttcaagacaaccattccattaatgaagcattttataggcacccgttctttatagtagagaaagatggccaaaatgactctcaaatacatttttactacacatgatatttcagcacagatgagtagtaggtcttcaggcaaaattgctatgctattatcctatgagttggaaggttacataagaagattagacatttatagaaataaaacatgcccattaaaagtgggcaaaggacatgaacagacactatatacagatactatttgtaagcgactcaagttggctgttgttttcttcctcatttgaaatactgtattgaagacttcataactcacatcatctctctgttggcccctttttcctggacaaatagaaatatgagaaaacggtctgaggatcatgctgtaacaaagtgttttatatcagctttacttgaccactcatgcttatgcccttgtagcatcttatccattgggaaaatgttcccagctgaaaacagtcaattgcagctgcaattttaaacatggaatgcaatagatagactttaagaagttagtaaaatctctaaaactatatgcaaaaatttctctgcatgcttatatgcttgcttttctgaggaatagaagcttttattaactgttgtatttaaaaatttcaattaggccgggcttggtggttcatgcctgtaatcccagcactttagggaggccgaagcaggtggattgatcacctaagggtatgagttcaagaccagcttggccaacatggtgaaatccccgtctctaccaaaaatacaaaaaatttgctggacatggtggcgtgcacctgtaatcccagctactctggaggctgaggcaggagaattgcttgagcctaggaggcagaggttggagtgagctgagatcacaccattgcactccagcctgggtgacaagagtgaatctccatctcaaaagaaatttttttaaattaattattatttgttgagacagggtctcactttgttgcctgagctgtgggcagtggtgcaatcatggttcactacagcctcaacctcctggctcaagtgatcttcctgcctccacctccccagtagctgggacttacaggcgtgcatcaccacaccaggcaaattctttttttaatgttttgtagagacgaggccttgctacgttgcttaggctggtctcaaactcctgggctcatgcaattctcctatcttagcatctcgaagaggtaagataaaaggcatgagccacttcacctggcctcattaacttttcaaaggagtatataagtttaaaaagtcaggatccagtgatgtaaaactttagtttccttccctgttgccattgagctcctccttctccctttctcctccccctccttctccttcttctccttcttcctcttcctcctccacctcctcctcttcttcttctcctcttcctcctgcttctcctcttcctccccccctcccctcctccccttcctcctcccctcctccctctcttccccctcctccccgcctcctcctccccctcctcctccccctcctcctcctcctccccctcctcctcctccccctcctcctcctctttcttcttctttttctttctcttcttcttcctcctcctccccagtcgtcatcttcttcttccttttcttcttctccttcctcctcttctccttctccctcctcctccttcttccttcttccttctcttcttcttcttcttatcctcctcctccttcatcttcctcctcctcctcttcttctcctcctccttatccttcctcctcccccctcctcttcttcttcttcttctccttctccttcttcttcctcttcttcttcctcctcctcccccctcgtcgtcatcttcttcttcttccttttcttcttctcctttctcttctccttctccctcctcctccttcttcttccttttcttcgtcttcttctccttcctcctcttctccttctccctcctcctccttcttccttcttccttctcttcttcttcttcttctcctccttctccttcctcctcttcctcctcctcttcctcttcctcttcttctttcttccttttttactttgccatagagacaatactttaggttcactaaaatttttgcagttgtgggatattgattcaagcacctgctcatttaaagtggttggctatatttaaaatgtttttattgctttttatctcatacccccaaactgtgcatttcatctccaatttcattattggttgaattccacagccacaaatcatatgacttaaaatcttctaacttagttattaattttattgaagatttcctttaggggtactctggttttataaatagactaatatgacacaattcctacttaaaaactattttaaatttttattttaatagtttttgggaaacaggtggttttcaattacattaatgaatttttttgtttgcagatgatgcaatctatttttttaaattattatattttaagttctgggttacatatgcaggtttgttacataggtaaacatgtgccaggtggtttgctgcacctatcaacccatcacttaggtattaagcccagcatgcaataggtcttttccctactgctctccccacccgccctcccctgaaaggcctctgtgtgtgttgttcccctctctgtgtccagatgttctcatggttctgctcccacttataagtgataacatgtggtgtttgattttctgttcctgtgttagttt

SEQ ID NO: 2 - DNA, 6001 bp, Homo sapiens
caaggtaaaggttaggaatgaataaagaagcagtctgggggcgggagccacgtgctagttactggcagttggatttaaaggggacttacaaataaaatatcagcgcaggaagggcttctggcctttctggttccaagccactaattggacagatgaggaaagctgaggcctatggtggtggggaggttagtggcagagccagggctagattctttctccacatagcactgttatcttgggttgaatcttgacaggtcctgtaccacacttctctttcataataataggacttgccaattggtgtagctcttttatattctttattgaatcctcacaactctttatgtatccaggcaagggttgttgtcttcattttgcagacaaagaatcagggtctcatagggatgaatgatccagagctgattcgcatgtacagtgtaattgagcacatgaattcataggtgacactgcccgagtctgtgctgtcctccctgcctggaaagtcactcacccctacctcttctatagttcaagggctacctccttcaacaatcattttctgaattctcctgcatgagttgggtgaccctctaggaggaaactgcctcttctggatgccgaggtcctccagaggggcagacaccttggcatcccacctcatggtggaagctgggagcccctagccaggcgcagccacctcctgtgcctgggatgaagtccactccgttctttcactccattatactgcatttgccacacccttctgagaccgcaggcttcttgaaagcagagtttatgtcttccatttttgagcaccaaagtctggtttcttggtggtattttgcaaatgtatgttgactcaatcataaactttttagagtcaggattttaacacaggtctcctgatgccatcaacttctgctccttttaggactgtgagttatagaactagaaagtaggaaaacgcctctcctgggtatagattaaccatccatgaaggccaatcttttccaattacaaataaagccacccaaaagcacaccagctctgctcctcccagtcttacaacaaaaccttatcagggaagagtctctgaatattttttatttcactgtgcacgagctggaaaaatgagcctccaagcctcagggaagcttggagagtgcttagaaagagtgccgacagtcacttacatctcagatatgagaaaaagacaaaagagacaagtgataaaaatagagcaaatgagggaactagagagacttgtttggcgagtggtggcaaccagtgttctgtggatgtggacggttcctggtcatcaaagttatggtcagctctggttcctgttcgcctggacccagacttaccaaggaagaaaacatttacctttagcatgtgcaagggccgggctgcactgcctttttctaaattctctcaccagaatctacaatgaaattaatgcttctctaattatttcaggcatgtctgagtggcagtgacttatcaaccccatagatttggccactgtctcattttatgtccatgtgtgggagttttgatttataggacttatccttgagcaaaagatttcttgtgtcatagcaggaccagggccaggaaaaactggtggtgagatctgagagcactggggagaaatagtctcttctagtactgttatatgatggtttcccataacttctttttttttttttctcaagatggagttttgctctgtcacccaggctggagtgcaatggcgcaatcttgactcactgcaacctccgcctcctgggttcaagcgattctcctgcctcagcctcccgagtagctaggattacaggtgcgcaccaccatgcccagctaattttttgtatttttagtagagacggggtttcaccatgttggccaggctggtcttgaactcctgacctcatgatctgcctgccttggcctcccaaagtgctgggattacaggcatgagccaccctgcccagccaattttccataacttcttttttttttttttgagacagagtctcgccctgtcgcccaggctggagtgcagtgatgtgatcgtggctcactgcaacctccacctcccgggttcaagcgattcttctgcctcagcctcccgagtagccgggaccacaggtgcgtgccaccacgcccggctaatctttgtagtttttagtagagacggggttttaccatgttggccaggcagatcttgaacttctgactttgtgatccacccacctcggcctcccaaagtgctgggattataggcatgagccaccgcacccggccctggttttccataacttctaatgtgtagagttccattggattttaaaagaaatgatgcttttttaaaagcatcattttaaaagcattaaaagaaatgatacatgcttattatacaaaatcaagtgatacatgatggtacaggaaaaaatatttaaaaattctaccacacacacacaacgtttggtgacatttggtgaacatcctttcagatactatgtgtatgtattggaagacaaaagaatggctagaataattggaaaaatgggctcctgcgacttattccattttagaaaaacttaagtcaattaacacattttaattggcaaataaggaactaaaatgaattcactttagtgacatagtttttctagaatttaaaaacgttcatcacattcttttaaattttacatttttaagaaggataaaatggcctgtaaatgccaggatttttggaatcgcaggactggaaagaaaagcaacgctgagccacttgttgcagatttttgcttttagggaggacaacggcagagtgctgagcttgtttttgaagtattcttaaagaacgcgagggatgaagcctccaactgcttcctcagtaaatggccaacatttactgatccttttggttcacaagcatttcacttgcaatatggtttcaggatttcttttggtatttctgtagagtgcaaattgttcagaatcaaaatcattctgctttgcatgattttctggctgaaacatttgtagttttcacatttgattcatctttctgatgaactcagttgtcgtcttgaaagggagtgggtactttgtaagacagctcagctgttgagtgaatgcatggggttgcatgtgggtgtgttggggtcagaagggagccctgtcagaaggtagctgtcatgaactgggtacaccgtctgtttgtgtgccttttcaagacaaccattccattaatgaagcattttataggcacccgttctttatagtagagaaagatggccaaaatgactctcaaatacatttttactacacatgatatttcagcacagatgagtagtaggtcttcaggcaaaattgctatgctattatcctatgagttggaaggttacataagaagattagacatttatagaaataaaacatgcccattaaaagtgggcaaaggacatgaacagacactatatacagatactatttgtaagcgactcaagttggctgttgttttcttcctcatttgaaatactgtattgaagacttcataactcacatcatctctctgttggcccctttttcctggacaaatagaaatatgagaaaacggtctgaggatcatgctgtaacaaagtgttttatatcagctttacttgaccactcatgcttatgcccttgtagcatcttatccattgggaaaatgttcccagctgaaaacagtcaattgcagctgcaattttaaacatggaatgcaatagatagactttaagaagttagtaaaatctctaaaactatatgcaaaaatttctctgcatgcttatatgcttgcttttctgaggaatagaagcttttattaactgttgtatttaaaaatttcaattaggccgggcttggtggttcatgcctgtaatcccagcactttagggaggccgaagcaggtggattgatcacctaagggtatgagttcaagaccagcttggccaacatggtgaaatccccgtctctaccaaaaatacaaaaaatttgctggacatggtggcgtgcacctgtaatcccagctactctggaggctgaggcaggagaattgcttgagcctaggaggcagaggttggagtgagctgagatcacaccattgcactccagcctgggtgacaagagtgaatctccatctcaaaagaaatttttttaaattaattattatttgttgagacagggtctcactttgttgcctgagctgtgggcagtggtgcaatcatggttcactacagcctcaacctcctggctcaagtgatcttcctgcctccacctccccagtagctgggacttacaggcgtgcatcaccacaccaggcaaattctttttttaatgttttgtagagacgaggccttgctacgttgcttaggctggtctcaaactcctgggctcatgcaattctcctatcttagcatctcgaagaggtaagataaaaggcatgagccacttcacctggcctcattaacttttcaaaggagtatataagtttaaaaagtcaggatccagtgatgtaaaactttagtttccttccctgttgccattgagctcctccttctccctttctcctccccctccttctccttcttctccttcttcctcttcctcctccacctcctcctcttcttcttctcctcttcctcctgcttctcctcttcctccccccctcccctcctccccttcctcctcccctcctccctctcttccccctcctccccgcctcctcctccccctcctcctccccctcctcctcctcctccccctcctcctcctccccctcctcctcctctttcttcttctttttctttctcttcttcttcctcctcctccccagtcgtcatcttcttcttccttttcttcttctccttcctcctcttctccttctccctcctcctccttcttccttcttccttctcttcttcttcttcttatcctcctcctccttcatcttcctcctcctcctcttcttctcctcctccttatccttcctcctcccccctcctcttcttcttcttcttctccttctccttcttcttcctcttcttcttcctcctcctcccccctcgtcgtcatcttcttcttcttccttttcttcttctcctttctcttctccttctccctcctcctccttcttcttccttttcttcgtcttcttctccttcctcctcttctccttctccctcctcctccttcttccttcttccttctcttcttcttcttcttctcctccttctccttcctcctcttcctcctcctcttcctcttcctcttcttctttcttccttttttactttgccatagagacaatactttaggttcactaaaatttttgcagttgtgggatattgattcaagcacctgctcatttaaagtggttggctatatttaaaatgtttttattgctttttatctcatacccccaaactgtgcatttcatctccaatttcattattggttgaattccacagccacaaatcatatgacttaaaatcttctaacttagttattaattttattgaagatttcctttaggggtactctggttttataaatagactaatatgacacaattcctacttaaaaactattttaaatttttattttaatagtttttgggaaacaggtggttttcaattacattaatgaatttttttgtttgcagatgatgcaatctatttttttaaattattatattttaagttctgggttacatatgcaggtttgttacataggtaaacatgtgccaggtggtttgctgcacctatcaacccatcacttaggtattaagcccagcatgcaataggtcttttccctactgctctccccacccgccctcccctgaaaggcctctgtgtgtgttgttcccctctctgtgtccagatgttctcatggttctgctcccacttataagtgataacatgtggtgtttgattttctgttcctgtgttagttt

SEQ ID NO: 3 - DNA, 20 bp, Homo sapiens
gtggtgaaggatagggagat

SEQ ID NO: 4 - DNA, 24 bp, Homo sapiens
ccaaaaaataaaacaaaactcaac

(19) United States
(12) Patent Application Publication
(10) Pub. No.: US 2015/0044673 A1
(43) Pub. Date: Feb. 12, 2015

(54) DIAGNOSIS OF STEATOHEPATITIS

(71) Applicant: MEDIZINISCHE UNIVERSITAT GRAZ, Graz (AT)

(72) Inventors: Kurt Zatloukal, Graz (AT); Karl Kashofer, Graz (AT); Michael Trauner, Vienna (AT)

(21) Appl. No.: 14/368,227

(22) PCT Filed: Dec. 21, 2012
(86) PCT No.: PCT/EP2012/076678

§ 371 Date: Jun. 23, 2014

(30) Foreign Application Priority Data
Dec. 23, 2011 (EP) 11195537.3

(51) Int. Cl. C12Q 1/68

(52) U.S. Cl. CPC C12Q 1/6883; C12Q 2600/154
USPC 435/6.11

(57) ABSTRACT

The invention discloses the use of the methylation of the keratin 23 (KRT23) gene as a marker for distinguishing between steatosis and steatohepatitis.

The invention relates to biomarkers for distinguishing steatohepatitis from steatosis.

Fatty liver diseases comprise a spectrum of severity ranging from simple steatosis over steatohepatitis to cirrhosis and hepatocellular cancer (HCC). There are two major etiologies for fatty liver disease, namely alcohol and metabolic disorders such as obesity and type 2 diabetes mellitus (T2DM). Due to its high prevalence and potential for severe hepatic outcomes such as liver cirrhosis and HCC in a substantial fraction of affected individuals, fatty liver disease has become a major issue for the society and health care. Up to 30% of the general population is affected by non-alcoholic fatty liver disease (NAFLD), reaching up to 70% among diabetic patients. The prevalence of steatosis and steatohepatitis in obese patients undergoing bariatric surgery is as high as 76% and 37%, respectively. Steatohepatitis develops in about 20% of alcoholics and up to 50% of T2DM who are also obese (BMI>30). This places fatty liver disease as the most common liver disease of the 21^{st} century accounting for the majority of liver cirrhosis and HCC in Western countries. Its prevalence is expected to further rise in light of the ongoing epidemic of diabetes and obesity.

While simple steatosis has a relatively benign course and is principally reversible, steatohepatitis carries a poor prognosis and can lead to severe liver damage with progression to cirrhosis and HCC. Conventional non-invasive markers such as serum transaminases correlate poorly with the risk of development as well as progression of liver disease, and currently available routine liver tests may even be unremarkable in a significant proportion of patients with steatohepatitis. Therefore, in current standard clinical practice, non-invasive serum and imaging markers do not allow the distinction of relatively benign fatty liver from progressive steatohepatitis. This situation results in underdiagnosis and undertreatment of these disorders. The development of efficient diagnostic, prognostic and therapeutic strategies has been substantially hampered by the fact that the understanding of the molecular pathogenesis of steatohepatitis is still incomplete. Several studies showed that the different forms of steatohepatitis (alcoholic—ASH, non-alcoholic—NASH) cannot be morphologically distinguished, which suggests a common pathogenetic mechanism despite different etiologies of the disease. According to current concepts, deregulation of energy metabolism may lead to simple steatosis, which needs to be accompanied by a second inflammatory insult to lead to steatohepatitis. A major unsolved problem is the marked difference in the individual risk to develop steatohepatitis and to progress to cirrhosis (e.g., only 20% of heavy drinkers or 50% of obese type II diabetic patients develop steatohepatitis; Hispanics and Caucasians are more susceptible than Afro-Americans). However, the factors responsible for disease progression across the spectrum of fatty liver disease are poorly understood. Why some patients are protected against developing steatohepatitis or simple steatosis, while others are not, is still unclear. It is currently even debated whether steatosis and steatohepatitis represent two consecutive disease stages; alternatively, individuals may a priori be predetermined to develop either a rather benign steatosis or prognostically dismal steatohepatitis.

Diagnosis of steatohepatitis is difficult and currently only possible by liver biopsy (for example, NASH refers to findings on liver biopsy in patients with steatohepatitis in the absence of significant alcohol consumption). Therefore, liver biopsy remains the only means of assessing the presence and extent of specific necroinflammatory changes and fibrosis in steatohepatitis. However, firm recommendations of when to perform a liver biopsy in the routine clinical setting have not yet been developed. The use of surrogate markers, such as aminotransferases and fibrosis markers, are not adequate for monitoring diseases as is necessary for clinical studies. In US 2009/0304704 A1 various expression markers have been suggested for diagnosing a NASH disease state in a patient comprising determining a level of expression of a panel of more than 30 genes associated with the onset or progression of NASH in a patient sample and comparing the level of expression (an expression “profile”) with a predetermined value for NASH-associated gene expression in this panel of genes and correlating the level of expression with a NASH disease state. Such profile determination is complicated and difficult to establish in routine diagnosis and testing large numbers of samples.

Rodríguez-Suárez et al. (Proteomics—Clin. Appl. 4 (2010), 362-371) and Feldstein et al. (Hepatol. 50 (2009), 1072-1078) disclose the proteomic analysis of fatty liver disease. Bragoszewski et al. (Acta Biochim. Pol. 54 (2007), 341-348) disclose a gene expression analysis to distinguish steatosis from steatohepatitis. WO 2004/055520 A1 discloses a method of diagnosing NASH. WO 2010/045470 A2 discloses determining the risk for developing a hepatic disorder by determining the level of expression of certain makers.

There is still a need for further, single markers which can be used instead of profile determination for diagnosing steatohepatitis, especially for distinguishing between steatosis and steatohepatitis.

Moreover, it is also desirable, if such single markers are not only useful in biopsy material but also in non-invasive specimen, such as body fluids which can be easily taken from patients, such as blood or urine, or smears from various mucosae.

Accordingly, neither suitable tissue sample testing nor a suitable serum diagnostic tests which can easily be transformed to a routine method are available. This represents a huge unmet medical need (US 2009/0304704 A1).

It is therefore an object of the present invention to provide a suitable biomarker which allows a reliable, non-invasive, diagnosis of steatohepatitis, especially differentiation between steatosis and steatohepatitis. The present invention is specifically aimed to provide means and methods for differentiation between the clinically “benign” simple steatosis and steatohepatitis that might progress to liver cirrhosis and liver cancer.

Therefore, the present invention provides the use of methylation of the keratin 23 (KRT23) DNA as a marker for distinguishing between steatosis and steatohepatitis.

It was shown with the present invention that the methylation of the KRT23 gene is a highly reliable molecular marker for distinguishing steatosis from steatohepatitis. Moreover, methylation of the KRT23 gene can also be used for monitoring the development from steatohepatitis to HCC. KRT23 methylation can also be used together with other markers for steatohepatitis and/or HCC for further optimising diagnosis of such liver diseases.

The human KRT23 gene (2 alternative transcripts: NC_{—}000017.10, NT_{—}010783.15; Seq. ID. No. 1) is located on chromosome 17 (17q21.2; reverse strand) and spans from nucleotide 39,078,952 bp to 39,099,836 bp (chr17: 39,078,952-39,099,836). It has therefore a size of 20,885 bases. In the method according to the present invention, methylation status of the KRT23 DNA can be determined by determining presence or absence of methylation at any CpG motif (a “CG” dinucleotide) in the gene sequence. It is preferred, however, to determine presence/absence of methylation in a region containing more than one CpG motif, preferably in a region with at least 5, more preferred with at least 10, especially with at least 20 CpG motifs. Specifically preferred regions for determining the methylation status of the KRT23 DNA according to the present invention are the exon regions. The KRT23 gene contains 9 exons:

| Exons | | | Coding for Iso 1 |
|---|---|---|---|
| Identifier | Position on gene | Length | ENST00000209718 |
| ENSE00001844668 | 215-289 | 75 | |
| ENSE00000863452 | 682-1427 | 746 | Met 1-Gln 132 |
| ENSE00002367885 | 6180-6262 | 83 | Ile 133-Lys 160 |
| ENSE00000721520 | 7539-7695 | 157 | Lys 160-Gln 212 |
| ENSE00002370836 | 9028-9189 | 162 | Glu 213-Gln 266 |
| ENSE00002350831 | 9275-9397 | 123 | Ser 267-Thr 307 |
| ENSE00002358052 | 12061-12281 | 221 | Lys 308-Gly 381 |
| ENSE00002369459 | 13132-13163 | 32 | Gly 381-Val 392 |
| ENSE00001823724 | 14552-14939 | 388 | Val 392-Ala 422. |

A preferred region where methylation is determined according to the present invention is the promoter region defined as the region 6000 bases upstream of the transcription start (from nucleotide 39,093,836 to nucleotide 39,099,836 (chr17: 39,093,836-39,099,836 (Seq. ID. No. 2).

The determination of the degree of KRT23 methylation is specifically suitable for diagnosing NASH, since KRT23 methylation levels are significantly decreased in NASH patients compared to healthy individuals or individuals having steatosis. A demethylation of the KRT23 DNA (compared to a KRT23 DNA from a healthy person (i.e. a person not having steatohepatitis)) is therefore indicative for steatohepatitis. Demethylation is specifically present if 10% or more, preferably 20% or more, especially 30% or more of the methylated CpG motifs of the healthy KRT23 DNA region determined are demethylated in the DNA in the sample.

The method according to the present invention is advantageous compared to current diagnosis (grading, staging) of steatohepatitis which relies on liver biopsy as a diagnostic gold standard for differentiation between “simple” steatosis and (N)ASH. Routine biochemical serum tests (liver function tests) currently available underestimate the occurrence and severity of steatohepatitis. Although the degree of liver fibrosis may be a crude predictor for the development of liver cirrhosis, more sophisticated individual risk markers/profiles for the development of cirrhosis and hepatocellular cancer are required. Moreover, the relative contribution of cardiovascular versus liver-related morbidity/risk may vary significantly among individual NAFLD/NASH patients. Given the high prevalence of steatosis and steatohepatitis in the general population, there is an urgent need for diagnostic tests and prognostic biomarkers (especially for such markers which can be diagnosed in a non-invasive manner, i.e. without the need for liver tissue samples) which predict the individual disease course and need for and response to therapy

A specific aspect of the present invention relates to a method for diagnosing steatohepatitis in a sample of a human body fluid or a human tissue sample comprising the determination of the methylation of the KRT23 DNA in this sample and diagnosing steatohepatitis, if the methylation of the KRT23 DNA is decreased compared to a sample of this human body fluid or tissue sample from a person with no steatohepatitis.

Preferably, the body fluid is blood or a blood derived sample, preferably a serum or a plasma sample. This makes the present method a suitable non-invasive method which can be used even without previous taking of biopsies, especially liver biopsies which may constitute a certain risk factor. Measuring KRT23 methylation in DNA in blood or a sample derived from a blood sample is easily adaptable to larger numbers of samples and may therefore be established for routine testing, preferably for determination of the methylation degree of the KRT23 gene in free circulating DNA in blood, especially in plasma or serum samples.

On the other hand, the present method can also routinely be applied for tissue samples, especially liver tissue samples (e.g. taken from liver surgery) or liver biopsy samples (e.g. taken by usual liver biopsy needles, historical samples or samples from necropsies).

Determination of the methylation of the KRT23 gene is easily possible for a person skilled in the art. Both, preparation of DNA in the sample for determination of methylation as well as establishing the amount/degree of methylation are routine methods in the present field of technology. For example, there are several commercially available kits for extracting DNA from blood samples, as well as kits for determining the methylation of DNA (e.g. using the bisulfite treatment with subsequent PCR amplification).

The present invention refers to the use of the (determination of the) methylation (degree) of the KRT23 gene in a method for diagnosing steatohepatitis.

KRT23 is a gene/protein which is widely known and investigated; determination of the degree of methylation of the KRT23 DNA is therefore easily possible by using standard techniques well available to a person skilled in the art.

DNA methylation of the KRT23 gene can be detected and quantified by any method commonly used in the art, for example, methylation-specific PCR (MSP), use of restriction enzymes with activity governed by methylation status, use of antibodies specific for methylated nucleotide bases, polynucleotide sequencing, bisulfite treatment and sequencing, pyrosequencing, and absolute quantitative analysis of methylated alleles (AQAMA). MSP is a technique whereby DNA is amplified by PCR dependent upon the methylation state of the DNA. Determination of the methylation state of a nucleic acid includes amplifying the nucleic acid by means of oligonucleotide primers that distinguish between methylated and unmethylated nucleic acids. MSP can rapidly assess the methylation status of virtually any group of CpG sites within a CpG island, independent of the use of methylation-sensitive restriction enzymes. This assay entails initial modification of DNA by sodium bisulfite, converting all unmethylated, but not methylated, cytosines to uracils, and subsequent amplification with primers specific for methylated versus unmethylated DNA. MSP requires only small quantities of DNA, is sensitive to 0.1% methylated alleles of a given CpG island locus. MSP eliminates the false positive results inherent to previous PCR-based approaches which relied on differential restriction enzyme cleavage to distinguish methylated from unmethylated DNA. This method is very simple and can be used on small amounts of samples. MSP product can be detected by gel electrophoresis, CAE (capillary array electrophoresis), or realtime quantitative PCR.

Bisulfite sequencing is widely used to detect 5-MeC (5-methylcytosine) in DNA, and provides a reliable way of detecting any methylated cytosine at single-molecule resolution in any sequence context. The process of bisulfite treatment exploits the different sensitivity of cytosine and 5-MeC to deamination by bisulfite under acidic conditions, in which cytosine undergoes conversion to uracil while 5-MeC remains unreactive.

The level of DNA methylation may be represented by a methylation index as a ration of the methylated DNA copy number to the sum of the methylated DNA copy number and the unmethylated DNA copy number, a ratio of the methylated DNA copy number to the unmethylated DNA copy number, or the like.

If the level of methylation at the KRT23 gene in the test sample is lower than that in a normal sample, the subject is likely to be suffering from steatohepatitis. As used herein, a “normal sample” is a sample prepared from a normal subject, especially a normal body fluid, normal blood or blood derived sample, such as a serum or plasma sample.

Preferably, the KRT23 DNA is free circulating DNA in blood, especially in plasma or serum samples.

Preferred methylation determination methods are methods employing the bisulfite treatment.

Usually, the methylation degree of the KRT23 DNA in the sample is compared with a reference value for this amount with a known status concerning steatohepatitis and/or steatosis, i.e. a value which is known to be a healthy value (i.e. a value not affected by steatohepatitis) or which is known to be a diseased status with respect to steatohepatitis/steatosis, preferably of a given/defined stage of steatohepatitis.

Such reference, standard or control samples are all useable in principle for comparison with the sample of unknown status concerning steatohepatitis and/or steatosis diagnosed according to the present invention. Such reference, standard or control samples can be taken e.g. from a human subject with negative diagnosis concerning steatohepatitis and/or steatosis or undetectable ongoing steatohepatitis development. If such a control sample, standard sample or reference sample is said to be comparable to the sample that is taken from a human subject being suspected to be afflicted by ongoing steatohepatitis development according to the present invention, this means that both samples have been derived and treated equally. Thus, the sample in both cases may e.g. be a blood derived sample which has been further treated in a way to allow for determination of methylation of the diagnostic marker gene as mentioned above.

Blood plasma is the yellow liquid component of blood, in which the blood cells in whole blood would normally be suspended. It makes up about 55% of the total blood volume. It is mostly water (90% by volume) and contains dissolved proteins, glucose, clotting factors, mineral ions, hormones and carbon dioxide. Blood plasma is prepared by spinning a tube of fresh blood in a centrifuge until the blood cells fall to the bottom of the tube. The blood plasma is then poured or drawn off. Blood plasma has a density of approximately 1.025 kg/l. Blood serum is blood plasma without fibrinogen or the other clotting factors (i.e., whole blood minus both the cells and the clotting factors).

The diagnostic methods according to the present invention may also be carried out in saliva, bladder washing, semen or urine samples, especially urine samples.

Although the diagnostic fine tuning for established clinical practice will work with sequence listings for defining the methylation differences in the part of the KRT23 gene for which methylation status has been determined according to the present invention, it is also preferred to establish the diagnostic system with the comparison of the sample to be analysed according to the present invention and a sample of the same source of a known status. It is therefore preferred to use the methylation degree in the KRT23 DNA in a healthy sample and compare these to the sample under investigation. If the methylation of the KRT23 DNA is significantly reduced, diagnosis of steatohepatitis is indicated. The samples are preferably blood, plasma, serum, urine, semen or saliva samples: also here suitable comparison values are derived from samples blood, plasma, serum, urine, semen or saliva, respectively, from a patient with has a healthy status, preferable samples taken (earlier) from the same patient. On the other hand, the comparison may also be taken to establish known methylation patterns of KRT23 DNA in the sample taken. As usual in human medical diagnosis, absolute limiting value can be defined for each of the samples to determine difference between healthy (steatosis) and steatohepatitis and between different stages steatohepatitis or even HCC. These absolute values have to be defined and carefully confirmed depending on the very method applied for determining methylation of KRT23 DNA and the status of (pathology-indicating) demethylation. For the examples according to the present invention, a reliable test system has been established.

The level of methylation of KRT23 DNA assessed according to the present invention is measured and is compared with the level of expression of methylation of KRT23 DNA from other samples. The comparison may be effected in an actual experiment; or intellectually (by comparison with known reference values); or virtually (e.g. automatically in silico). When the methylation level (also referred to as methylation pattern or methylation signature (methylation profile)) is measurably different, there is according to the invention a meaningful (i.e. statistically significant) difference in the level of methylation. Preferably the difference in methylation of KRT23 DNA is at least 5%, 10% or 20%, more preferred at least 30% or may even be as high as 50%, 75% or 100% (100% being a complete demethylation in the region wherein the methylation status has been determined; i.e. all methylation sites in the healthy (steatosis) DNA have been demethylated). The diagnosis is, of course, the more accurate, the more methylation sites are included in the determination of the methylation status. It is therefore preferred to include at least 5, preferably at least 10, more preferred at least 20, especially at least 30 methylation sites (i.e. sites which are methylated in KRT23 DNA of healthy persons) in the determination according to the present invention. Preferably, these methylation sites are consecutive methylation sites in the KRT23 gene. It is also possible to examine more than one region with consecutive methylation sites, e.g. in the promoter region and in one or more of the exons.

The methylation level for KRT23 according to the present invention is therefore reduced in a disease sample compared to a healthy, normal (steatosis) sample if preferably at least 5%, 10% or 20%, more preferred at least 30% or even 50%, 75% or 100% of the methylation sites are demethylated in the disease (steatohepatitis) sample. Whether KRT23 methylation level is decreased in a given detection method can preferably be established by analysis of a multitude of steatohepatitis samples with the given detection method. This can then form a suitable level from which the “decreased” status can be determined; e.g. by the above % difference or -fold change. A decreased methylation of the KRT23 gene is indicative for steatohepatitis.

Within the course of the present invention, it was shown that the methionine-metabolism is compromised in steatohepatitis. This pathway is linked to gene regulation through modification of methyltransferase-activity via changes in S-adenosylmethionine levels, leading to a modification of the activity of steatohepatitis-linked genes via alterations in histone- and DNA-methylation. As increased cell death rates are a key feature in advanced steatohepatitis, leading to the release of genomic DNA, DNA with such characteristic methylation signatures may be detectable also in the circulation.

These two observations now provide the rationale that the overexpression of KRT23 mRNA is due to altered DNA methylation and that altered methylation of the KRT23 promoter can be detected as free circulating DNA in serum or plasma.

A diagnostic assay for altered KRT23 methylation according to the present invention can be based on the detection of free circulating DNA the KRT23 promoter methylation in surgically resected or explated human livers (comparison of normal liver with simple steatosis and steatohepatitis). Based on the type of methylation patterns detected an assay for free circulating DNA can be established (e.g. as generally disclosed in WO 2008/103761 A2). This assay can be for example a methylation-specific PCR or sequencing of specifically enriched free circulating DNA that has been treated with bisulfite.

Accordingly, a diagnostic assay in serum using KRT23 methylation as a signature for steatohepatitis is provided according to the present invention.

Another important aspect of the present invention is a kit for determination of methylation of the KRT23 DNA in a sample for use in diagnosing steatohepatitis and/or for distinguishing between steatosis and steatohepatitis. The present inventionalso relates to the use of a kit for determining the methylation level of KRT23 DNA in a sample, comprising determining the amount of demethylation of the KRT23 gene compared to a healthy methylation pattern for diagnosing a tissue sample or a sample of a body fluid for steatohepatitis. The kit according to the present invention contains suitable methylation determination agents for KRT23 DNA methylation. Such agents are well available for a person skilled in the art and depend on the very method applied for determination of the methylation status (e.g. MSP, use of restriction enzymes with activity governed by methylation status, use of antibodies specific for methylated nucleotide bases, polynucleotide sequencing, bisulfite treatment and sequencing, pyrosequencing, and AQAMA; see above).

If the method applies a PCR step, the diagnostic kit for diagnosing steatohepatitis in a tissue sample or a sample of a body fluid according to the present invention comprises:

a KRT23 methylation determination primer pair, i.e. a KRT23 binding primer pair which defines a methylation region in the KRT23 gene, preferably a primer pair which is free of CpG motifs, especially a primer pair which is free of single nucleotide polymorphisms (SNPs) and,

optionally (if the method includes a bisulfite step), an agent containing bisulfite.

In general, the kit according to the present invention includes a KRT23 methylation determination primer pair which specifically binds to the KRT23 DNA and DNA amplifying reagents, such as a suitable PCR DNA polymerase and reagents (nucleotides, buffers, etc.) for performing PCR in connection with methylation determination. These primers do not contain CpG motifs and flank the CpG islands of which the methylation status is to be determined. The primers thus bind in the genomic region of the K23 gene (Seq. ID. No. 1) or in the promoter region of the KRT23 gene (Seq. ID. No. 2). Preferably, the primers are designed so as not to contain SNP loci (currently, the following 386 SNPs are known for the KRT23 gene (all sequences are included in Seq. ID. No. 1; reference is therefore made to the positions in Seq. ID. No. 1):

| | | | | | |
|---|---|---|---|---|---|
| 39081713(−) | CGCCAC/TGAACT | 39080117(−) | AAGTTC/GCAGGA | 39092868(−) | CCAAAC/TACAGA |
| 39084504(+) | CTGTGC/TCTGCA | 39080393(+) | GAGTGT/CTCAAC | 39092756(−) | CCACCG/ATCCAT |
| 39080910(−) | CTCCCG/ATGAAC | 39085145(−) | TGTACC/AACCAC | 39081546(−) | GTATAC/TGTGTG |
| 39080926(−) | TGTATC/GCAGAC | 39082641(−) | GGAGAG/ATAGGA | 39078939(−) | TCACGG/TTTTTT |
| 39080921(−) | CCAGAC/TGGGTT | 39087292(−) | AGTGAA/GACTCC | 39078825(−) | TCACGG/TCAGCC |
| 39084761(−) | ATGAGA/TCAAGA | 39094607(+) | NNNNCG/ACAGGC | 39083616(+) | TAAAAC/TGTTTT |
| 39082946(+) | AAAATG/ATATCT | 39078701(+) | CCTCCC/TTTTCT | 39082351(+) | TAGTCT/GCAAGG |
| 39086932(+) | AATCGT/CTTGAA | 39081892(+) | GATTGT/CTGAGT | 39081248(+) | GCATC-/TTTTTTT |
| 39091906(+) | GTCATA/GATGAC | 39087265(+) | ttttt-/TTTttttt | 39085894(+) | TCTCAC/TGCATT |
| 39091943(+) | TAGGCG/ATAATC | 39089145(+) | TCCGCA/GCTTAC | 39083057(+) | TTGAGC/ACCCCT |
| 39092063(+) | TGCACC/TGAGCA | 39095092(+) | AACTAG/AAGAGA | 39091364(+) | TTTTT-/TCATAA |
| 39086681(+) | ATTTTG/AAGACT | 39079744(+) | GAATGG/TGTGAG | 39092784(+) | CTGGGC/TCGGCC |
| 39092080(+) | ATGAAT/CACCAG | 39083232(+) | CATCAG/AACACA | 39082319(+) | TCTATA/GTTTTA |
| 39087401(+) | TGGGAC/TTACAG | 39089491(+) | TCCTGC/TCTCAT | 39083947(+) | TTCTGG/AGTGAT |
| 39093573(+) | TAGATG/ATAATT | 39094133(+) | GCCAAT/ATGGTG | 39091919(+) | ATATGC/TTGGTG |
| 39093883(+) | GGGAGC/TCACGT | 39088597(+) | TTATGG/ATTTGT | 39093291(+) | CAAAAC/ACAAAA |
| 39094685(+) | CAAATG/CTATGT | 39094468(+) | CCAGAA/GGGGCA | 39084313(+) | CCATCA/GGGACA |
| 39094687(+) | AATGTA/GTGTTG | 39092396(+) | ATTTTT/CCCTCC | 39088500(−) | AAGTC-/TTTAACT |
| 39094977(+) | CTCCAA/CGCCTC | 39093484(+) | GGTGGT/CGAATA | 39087028(+) | ACAAAC/TAAATA |
| 39083591(+) | AGGGCC/TATTGT | 39079891(+) | TTAAAC/TTAAAA | 39085440(+) | CAACAT/CAGTTA |
| 39083713(+) | CAGCAG/ATCATT | 39083534(+) | ATTGGC/TGTCAG | 39089224(+) | CAATAG/ATTACT |
| 39083795(+) | CAATTA/GTATGG | 39092160(+) | AAAGAT/CGTTTT | 39093299(+) | AAACAA/GAACAA |
| 39083875(+) | ATGAC-/ACATGC | 39078845(+) | ATGTCT/CGGAGA | 39093301(+) | CAACA-/ACAGCAAC |
| 39091714(+) | GGTATG/TTGCTT | 39093618(+) | TGCCTG/TTCATC | 39093298(+) | AAAAC-/AAAACAAC |
| 39081725(−) | CTGACG/ACAGCT | 39092597(+) | CTCCAC/GGTAGG | 39083936(+) | ACTTA-/AGAGTT |
| 39095706(+) | tttttA/Gtattt | 39092496(+) | TACTGG/TGAATA | 39079666(+) | CAGATC/TCCTTG |
| 39095702(+) | taattC/Ttttgt | 39084803(+) | GGACCC/TGTATC | 39087597(+) | AAACCC/GGAACA |
| 39095694(+) | tgcccA/Ggctaa | 39079332(+) | TTGCAG/AACACT | 39089548(+) | TTGCTA/GTAAAG |
| 39095682(+) | tgcgcA/Gccacc | 39089043(+) | CTGAT-/GCCCAT | 39090389(+) | GGTACG/AGGAGA |
| 39095681(+) | gtgcgC/Taccac | 39095679(+) | AGGTGC/TGCACC | 39090680(+) | ACCCCC/GCATTG |
| 39085280(+) | aaaaa-/AAaaaaa | 39082895(+) | TCTCT-/GGGATT | 39094309(+) | TGCCCG/AAGTCT |
| 39095535(+) | ttttt-/Tctcaa | 39092735(+) | GCGGCC/ATCCCC | 39094525(+) | CAGGCG/TCAGCC |
| 39095700(+) | gctaa-/Tttttt | 39089981(+) | ACTTAG/ATACAG | 39087810(+) | TTGTTA/GGTACA |
| 39095708(+) | ttgta-/Gttttt | 39095043(+) | ATGAG-/AAAAAG | 39082714(+) | GCTTCC/TTAATT |
| 39095307(+) | CTAATC/TATTTC | 39089237(+) | TCAAA-/GGAGTT | 39086569(+) | ATAAAC/TAGTCA |
| 39093885(+) | GAGCCA/CCGTGC | 39095206(+) | CAAGG-/GAAGAA | 39094560(+) | CACTCC/TGTTCT |
| 39094331(+) | CCTGCC/TTGGAA | 39095510(+) | TACTGG/TTATAT | 39091364(+) | CCTTGG/TTTTTT |
| 39093869(+) | AGCAGT/CCTGGG | 39094627(+) | CAGAG-/TTTTAT | 39089394(+) | ATTCAG/ATGACT |
| 39095288(+) | CAATGA/GAATTA | 39088500(+) | CAGTT-/AAAGACT | 39086014(+) | ATGTGC/ACTAGC |
| 39095359(+) | TGGCCA/GCTGTC | 39085652(+) | AACTGG/AGCTCT | 39084679(+) | GAAGTT/GTGTGC |
| 39089553(+) | ATAAAA/GAAGGA | 39085387(+) | TGCTT-/CCCATA | 39093667(+) | CAGGAT/CTTCCC |
| 39089358(+) | TACACT/CTTTCT | 39085352(+) | TTGAT-/CCCGTG | 39080276(+) | ATACAG/CTGAAC |
| 39089568(+) | TTTAGG/ATATTT | 39084491(+) | TCACCG/ATGCTG | 39083518(+) | ATCCTC/TAGTGC |
| 39093376(+) | AAGCCC/TCACCT | 39094712(+) | TTTTA-/GGAGTC | 39095289(+) | AATGAA/GATTAA |
| 39089796(+) | GAAAAC/TCAAAA | 39089091(+) | GAGATG/AGCATG | 39082393(+) | AAAAAT/ATGTCA |
| 39081966(+) | AGTAGG/TCTGAC | 39083010(+) | AGCTAC/GAGCAA | 39080452(+) | TAGGGG/ATTTAG |
| 39083729(+) | AAGTGC/TACCTG | 39092611(+) | CCAGGC/TGGTCG | 39085142(+) | GGTGTA/GGTTGT |
| 39093124(+) | CCGGAG/AGTGTG | 39081799(+) | GGTCTC/TGGATA | 39079972(+) | GACCCA/TTGTCT |
| 39078588(+) | CTTAGA/TGTAGC | 39082632(+) | CAGCCA/GGTCTC | 39083774(+) | TGAGGA/GTAGAA |
| 39087551(+) | GCCACT/CGCGCC | 39083250(+) | CTTGTC/TGCTTT | 39087025(+) | TAAAC-/AAATAAATA |
| 39080319(+) | CACACA/CGTATT | 39078686(+) | GTGAGG/TCTTTC | 39081638(+) | CGGTAC/TGTGGT |
| 39079961(+) | AGAGTC/GTCAAA | 39092741(+) | TCCCCC/ACGCAC | 39085511(+) | AGCAGA/TAAGAA |
| 39086013(+) | CATGTG/AACTAG | 39095194(+) | GGACCC/TAGACT | 39079555(+) | ACCTTC/TTGGAG |
| 39087171(+) | TAAACT/GAAGGT | 39095462(+) | AACTGG/TTGGTG | 39082911(+) | CACCAA/GCTTTC |
| 39091363(+) | ACCTTG/TTTTTT | 39090251(+) | ATTGAG/TAATCA | 39095807(+) | GATTAG/TAGGCA |
| 39084361(+) | TAAACA/GATTTG | 39085260(+) | GGTGAC/TAGAGT | 39087692(+) | GGTCAT/ACTTAC |
| 39089397(+) | CAGTGA/TCTACA | 39084556(+) | ATGTCA/GCCTTG | 39084543(+) | CTTCAG/ATTCGT |
| 39090615(+) | TTTCCT/CATTCA | 39090915(+) | CTACAC/TTGTAG | 39094656(+) | CCAAAA/GTCTGG |
| 39088006(+) | ATATAG/AGAAGG | 39086229(+) | CTTTCC/TTCATT | 39091664(+) | GATGAC/TTGGAT |
| 39086034(+) | AATTAG/TAAACA | 39082303(+) | CAACTA/GAACTG | 39085910(+) | TTATAC/GTAACA |
| 39087546(+) | TGTGAA/GCCACC | 39084901(+) | GACAAA/TCAGAA | 39090870(+) | TATTCA/CTACTA |
| 39095535(+) | ACTTCC/TTTTTT | 39086968(+) | CCGAGA/GTCACG | 39093741(+) | GCTGGG/TCTCTG |
| 39088053(+) | TTTAGG/ATTATG | 39085111(+) | TGTCTC/TTACTA | 39081780(+) | TGCAGG/AAGTAC |
| 39078590(+) | TAGAGA/TAGCCT | 39081614(+) | TCACTC/GTCTCC | 39093902(+) | TGGCA-/GGTTGG |
| 39088376(+) | TTTATG/ATCATT | 39081726(+) | GCTGCA/GTCAGT | 39081814(+) | GTTTTC/GCAAAG |
| 39092916(+) | GAACTC/GAGCCG | 39092514(+) | TTACTG/TCCAGG | 39088797(+) | GGCTGC/TATCAC |
| 39079976(+) | CTTGTC/TTTATT | 39082856(+) | CATCTC/TGGCAG | 39092584(+) | GGGCGC/TGAACC |
| 39093872(+) | AGTCTG/CGGGGC | 39083986(+) | TGCTTC/TGGAGT | 39083856(+) | CTTCAA/TAAATT |
| 39089403(+) | CTACAC/TTGTGA | 39083935(+) | AACTT-/AAGAGT | 39081730(+) | CGTCAG/ATTCCT |
| 39092410(+) | GGGTCG/CCCTGG | 39085661(+) | CTTTGA/CTGTTG | 39092707(+) | AGCTCC/AGCGTG |
| 39085062(+) | TAAAAG/TAAAGA | 39092640(+) | GCCTTC/TCCATT | 39084517(+) | TCAATC/TTCCAG |
| 39092215(+) | GCACTC/TCTATC | 39089292(+) | TTATCA/GGCATA | 39089040(+) | TCCACC/TGATCC |
| 39087559(+) | GCCCCG/ACCGAG | 39087562(+) | CCGCCA/GAGTTT | 39094487(+) | GGCATC/GCCACC |
| 39086728(+) | TTTAAC/AATAAA | 39092174(+) | TCTGTA/GAGAAA | 39092082(+) | GGCATC/GCCACC |
| 39087866(+) | TAAATG/ACTTAG | 39081760(+) | CTCTTG/ACATGT | 39083625(+) | TTGAAA/TGTTGC |
| 39095812(+) | CAGGCG/ATGAGC | 39081721(+) | GCGTAG/CCTGCG | 39094069(+) | TGTTAC/TCTTGG |
| 39091399(+) | GCACTA/GTATAG | 39092835(+) | TGGCTG/AAAGCT | 39090679(+) | CACCCC/TCCATT |
| 39084256(+) | TTACTG/ATAAGG | 39092668(+) | TGCTTC/TTTCCA | 39087685(+) | GAGCAT/CTGGTC |
| 39084536(+) | ATGTGC/TGCTTC | 39084606(+) | CATGGC/TTGCAG | 39085337(+) | CTCCAC/TGATTA |
| 39081639(+) | GGTACG/ATGGTG | 39087217(+) | ATGCC-/TTTTCA | 39078771(+) | CATGAA/CTCATG |
| 39083574(+) | TGCCTC/TGCCTG | 39081975(+) | ACTTAC/TAGGTT | 39082376(+) | ATGACA/GATGGT |
| 39083915(+) | TAGAAA/GTGACT | 39091203(+) | CTTCCA/GCGAGC | 39090151(+) | TTGGCA/GTATTC |
| 39093231(+) | GTCCAC/TCCAAA | 39081808(+) | TAACAT/AGTTTT | 39084152(+) | CACCAA/GTTACT |
| 39089048(+) | TCCCAT/CGGAAT | 39095299(+) | ATGCTA/TCTCTA | 39082165(+) | GCCAGA/GTAAAA |
| 39084649(+) | ATTGGG/AACCTC | 39094396(+) | TTTTCC/TGAATT | 39084728(+) | TCTCGA/TTGCTT |
| 39084638(+) | AAGGCG/ATCAGC | 39082270(+) | CATGC-/AAAAAA | 39091904(+) | ATGTCA/GTGATG |
| 39094038(+) | GCCAGA/GGCTAG | 39084513(+) | CAGGTA/C/TAATCT | 39082232(+) | TAACTA/GTGTCC |
| 39084264(+) | AGGATG/TAATGA | 39091959(+) | AATAGC/TGTAAC | 39080882(+) | TCTGAA/GCTTAG |
| 39081490(+) | GCTCAA/GCACAA | 39085019(+) | GTTGGG/TGATAA | 39093783(+) | AACAGC/GAAACC |
| 39094941(+) | TTTTTA/GTTTCA | 39091225(+) | TTGACC/TGGTTC | 39085336(+) | CCTCCA/GTGATT |
| 39086946(+) | GGAAGG/TCAGAG | 39095345(+) | CAACCC/GCATAG | 39085153(+) | ACACAC/TCTTTA |
| 39084825(+) | TGACAC/TTGAAG | 39085634(+) | TCTAAC/TCAGCT | 39088367(+) | GAAGGC/TGTGTT |
| 39090409(+) | ACTCAA/GAGCAT | 39088345(+) | TTGCCC/TGAAGT | 39089738(+) | CACTGA/GGGTTT |
| 39091206(+) | CCGCGA/GGCTCG | 39083017(+) | GCAAAA/GCTCCT | 39082806(+) | TGGGTA/GCTGGC |
| 39088264(+) | TTTCTA/GATCAG | 39092544(+) | TTCAGA/GATGCG | 39093284(+) | CAAAAA/CCCAAA |
| 39088625(+) | TTATAA/GTCAGA | 39085299(+) | AAAAAA/GAAAGA | 39086427(+) | TAGAGA/GTGAAT |
| 39085605(+) | CACAGA/GAAAGG | 39078657(+) | GCATAA/CTTTTT | 39080435(+) | ATTGCC/TTCTTA |
| 39089006(+) | AGGCAA/CCTTTT | 39082607(+) | GGCGGC/TTAAAG | 39093333(+) | ACATTA/GAACAA |
| 39082885(+) | TATCAA/GTATTC | 39093330(+) | CAGACA/GTTAAA | 39086301(+) | GGCCCC/TCGACT |
| 39094308(+) | CTGCCC/TGAGTC | 39093052(+) | GGGCCC/TTCGCA | 39083219(+) | TGCTCC/TTCCAA |
| 39081232(+) | CATCTC/GTAGCT | 39079289(+) | CTTCCG/ATTGAT | 39079816(+) | TGAAAC/TATCAA |
| 39079826(+) | ATAAAG/TCAATT | 39089921(+) | GAATTC/TTGAAA | 39086339(+) | TGTTCA/GTTTTC |
| 39086133(+) | AATCAC/GCCTGA | 39079847(+) | GGGAAG/TAATTG | 39092606(+) | TGTTCA/GTTTTC |
| 39093285(+) | AAAAAC/TCAAAA | 39086179(+) | GTTCCC/TGGGGA | 39092415(+) | CCCTGG/TCAAAG |
| 39095689(+) | CACCAA/TGCCCA | 39089063(+) | TATCAC/TGAATG | 39081702(+) | TCTGCC/TGCTCC |
| 39079207(+) | GAAAAC/TAGATT | 39086740(+) | AGGCCA/GGGCAC | 39092694(+) | CCAGGA/GGGTGG |
| 39092961(+) | CTGTGA/GGAGTT | 39080472(+) | TCCATC/TACTTA | 39084731(+) | CGATGC/ATTCTT |
| 39092277(+) | ACAAAA/GCAGAC | 39093684(+) | CTGGGA/GGAGGA | 39088286(+) | AACGCC/TAACAC |
| 39085193(+) | AGAATC/TGCTTG | 39084806(+) | CCTGTA/GTCCAC | 39093298(+) | AAAAC-/AAAACAAC |
| 39089336(+) | TTGTTA/CAGAAA | 39086370(+) | GAGAAG/TAGTGC | 39087055(+) | ATAAA-/TAAAATAAA |
| 39086623(+) | ATTTGC/TTTCAT | 39083177(+) | CTTCCC/TTTCAT | 39087676(+) | GAATAA/GTCTGA |
| 39087068(+) | TAACAA/CTACAC | 39081148(+) | GGATCA/TTAGAA | 39086182(+) | CCTGGG/TGAGTT |
| 39082507(+) | TGAAGC/TGCATA | 39093935(+) | AAATAC/TCAGCG | 39092689(+) | ACCCTC/TCAGGG |
| 39083133(+) | TCCTCA/CCTCTT | 39094816(+) | GGTATA/GGATTA | 39087024(+) | ATAAAC/TAAATA |
| 39093915(+) | TTAAAA/GGGGAC | 39088731(+) | GGACTA/GGAAAC | 39092687(+) | AGACCC/TTCCAG |
| 39081143(+) | CTTTCA/GGATCA | 39087806(+) | TGACTG/TGTTAG | 39081609(+) | ACCCTA/TCACTC |
| 39089989(+) | CAGAAC/TTATGA | 39081375(+) | CTTCTA/GGAGAC | 39086378(+) | TGCACC/TCATTG |
| 39093432(+) | AACAGA/GATCTC | 39089997(+) | TGATAC/TAACCA | 39092862(+) | GTCCCA/GTCTGT |
| 39088712(+) | TTACGC/GCAAGT | 39091205(+) | TCCGCA/GAGCTC | 39084635(+) | GCAAAA/GGCGTC |
| 39080468(+) | TGACTC/TCATTA | 39095680(+) | GGTGGA/GCACCA | 39080750(+) | CTTCCC/TGTGTC |
| 39089755(+) | TGACAC/TACACT | 39081587(+) | CTCTCA/GTGCCA | 39092709(+) | CTCCGC/TGTGGT |
| 39091111(+) | TGTGTA/TTTAAA | 39083617(+) | AAAACA/GTTTTG | 39092893(+) | CTGCTG/TCACTC |
| 39086354(+) | CTTTGA/GTGAGA | 39078826(+) | GCTGCC/TGTGAA | 39081573(+) | TCCCTC/TGCATG |
| 39084651(+) | TGGGAC/ACTCAT | 39091584(+) | AAATCA/GTGGTC | 39084804(+) | GACCTG/ATATCC |
| 39081548(+) | CACATA/CTACCC | 39089167(+) | GATTGG/TCTGAG | 39091379(+) | TTTTTC/TATAAA |
| 39083300(+) | CCACAA/TCAAGC | 39089970(+) | TATTCA/GGGCTT | 39081259(+) | TTTTTC/TTCT |
| 39081284(+) | AACACA/TTTCCC | 39092253(+) | ATCCAC/TGGACA | | |
| 39094526(+) | AGGCGC/TAGCCA | 39083112(+) | CTCCGC/TGGCCT | | |
| 39092491(+) | CCTCAC/TACTGG | | | | |
| 39081261(+) | TTTTTC/TTCTTT | | | | |
| 39084597(+) | CTCCTA/GGGACA | | | | |
| 39092808(+) | GCGCCA/CTGGAA | | | | |

Preferred primer pairs according to the present invention are designed to amplify a region in the KRT23 DNA which is at least 20 nucleotides, preferably at least 50 nucleotides, especially at least 100 nucleotides long (nucleotide count without the nucleotides of the primer themselves). However, the size can also be significantly longer; maximum lengths are defined only by the technical practicality of the PCR method (if the amplified sequence is too long for appropriate amplification and/or sequencing). According to a preferred embodiment, the primer pairs are designed to amplify at least 5, preferably at least 10, more preferred at least 20, especially at least 30, methylation sites of the KRT23 DNA (again the term “methylation sites refers to sites which are methylated in a healthy DNA). Methylation occurs at cytosine bases on the DNA, usually where the cytosine is followed by a guanosine base (therefore being a “CpG-motif”). Possible methylation sites are therefore all sites having the nucleotide sequence “CG”. This is valid for both DNA strand (therefore, a “GC” motif in the sense strand indicates a CpG motif in the antisense strand. Possible methylation sites in the DNA (being double stranded) are therefore all CG and GC motifs in Seq. Id. Nos. 1 and 2). Known and preferred methylation sites are indicated in bold in Seq. ID. No. 1.

A decreased methylation of the KRT DNA in the sample of the patient compared to the methylation in the KRT23 DNA in a sample of known steatohepatitis status, e.g. a sample from a healthy individual, indicates the steatohepatitis status of the patient. Such standard samples or other KRT23 standards are preferably further constituents of the present kit. In this embodiment of the invention, a gel electrophoresis or sequencing device or system detects the sequence which results from PCR amplification.

According to a preferred embodiment of the present invention, a kit for detecting the methylation level of KRT23 DNA in a sample is provided, comprising emulsion PCR beads (Williams et al., Nat. Methods 3 (2006), 545-550. Emulsion PCR isolates individual DNA molecules along with primer-coated beads in aqueous droplets within an oil phase. A PCR then coats each bead with clonal copies of the DNA molecule followed by immobilization for later sequencing. Emulsion PCR has been commercialized by 454 Life Sciences, is also known as “Polony sequencing” and “SOLiD sequencing” (developed by Agencourt, later Applied Biosystems, now Life Technologies) and allows amplification of complex gene libraries.

The kit can comprise suitable primer pairs specific for KRT23 DNA and/or reagents enabling methylation determination of KRT23 DNA in a given sample. The kit may also contain instructions for determining steatohepatitis diagnosis and prognosis based on the detection of the particular methylation degree or pattern according to the present invention. The present kits are specifically useful for diagnosing a tissue sample or a sample of a body fluid for steatohepatitis.

Such a kit may further include additional components such as additional oligonucleotides or primers and/or one or more of the following: buffers, reagents to be used in the assay (e.g., wash reagents, polymerases or internal control nucleic acid or cells or else) and reagents capable of detecting the presence of bound nucleic acid probe or primers. Of course the separation or assembly of reagents in same or different container means is dictated by the types of extraction, amplification or hybridization methods, and detection methods used as well as other parameters including stability, need for preservation etc. It will be understood that different permutations of containers and reagents of the above and foregoing are also covered by the present invention. The kit may also include instructions regarding each particular possible diagnosis, prognosis, theranosis or use, by correlating a combined KRT23 DNA methylation level with a particular diagnosis, prognosis, theranosis or use, as well as information on the experimental protocol to be used.

The present invention is further illustrated by the following examples and the drawing figures, yet without being limited thereto.

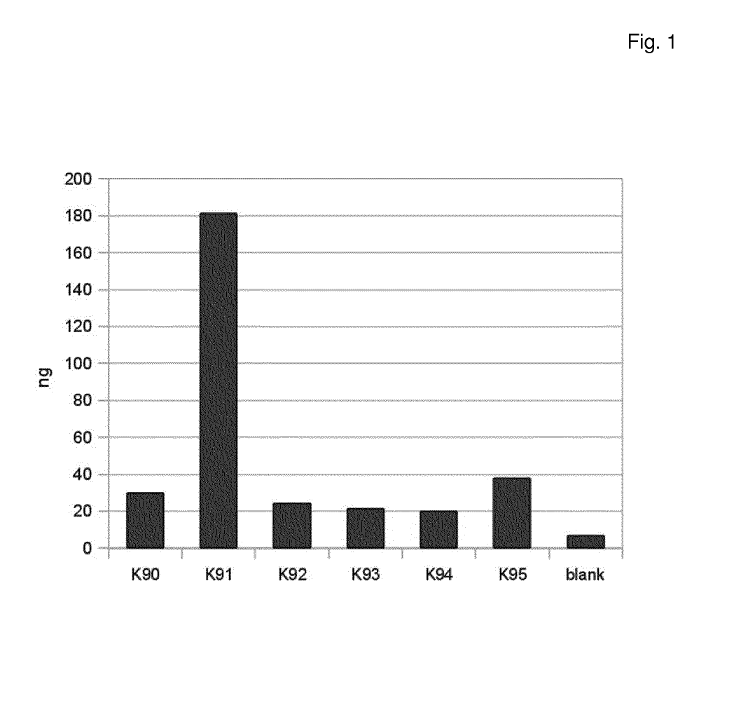

FIG. 1 shows DNA amount that can be extracted from 1 ml patient plasma.

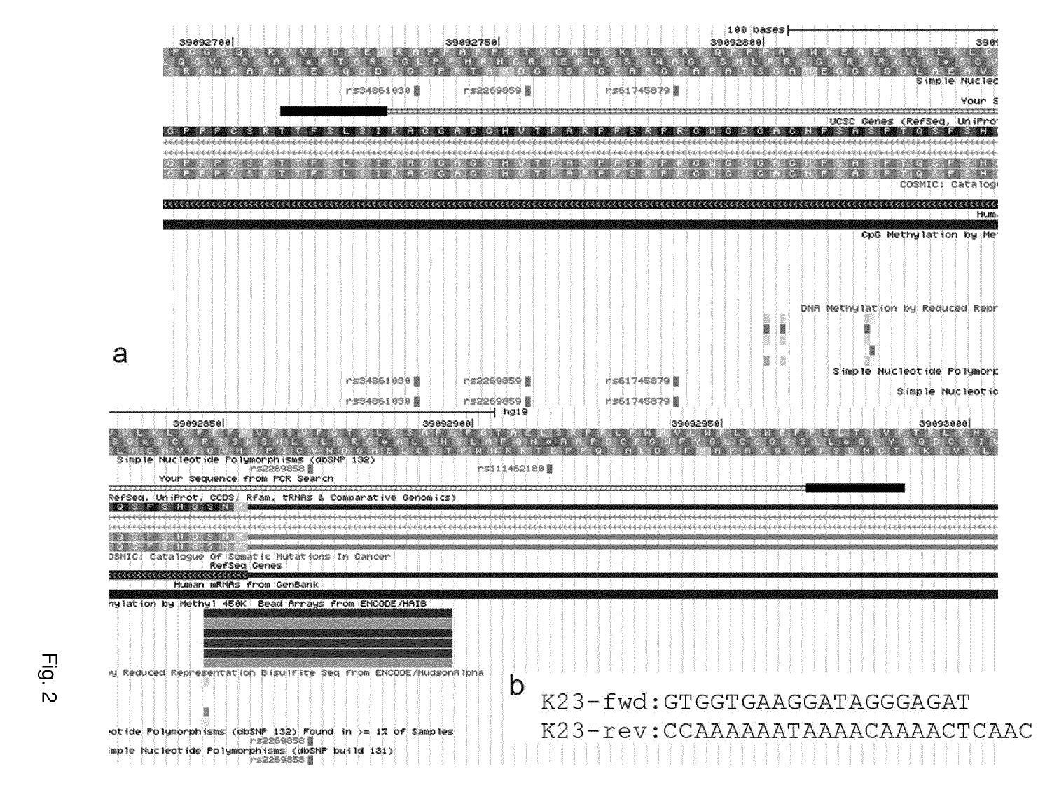

FIG. 2 shows the KRT23 gene including methylation sites in the first exon in region chr17: 39,092,678-39,093,032. Primers have been designed which include these methylation sites but do not contain CpG motifs or SNPs.

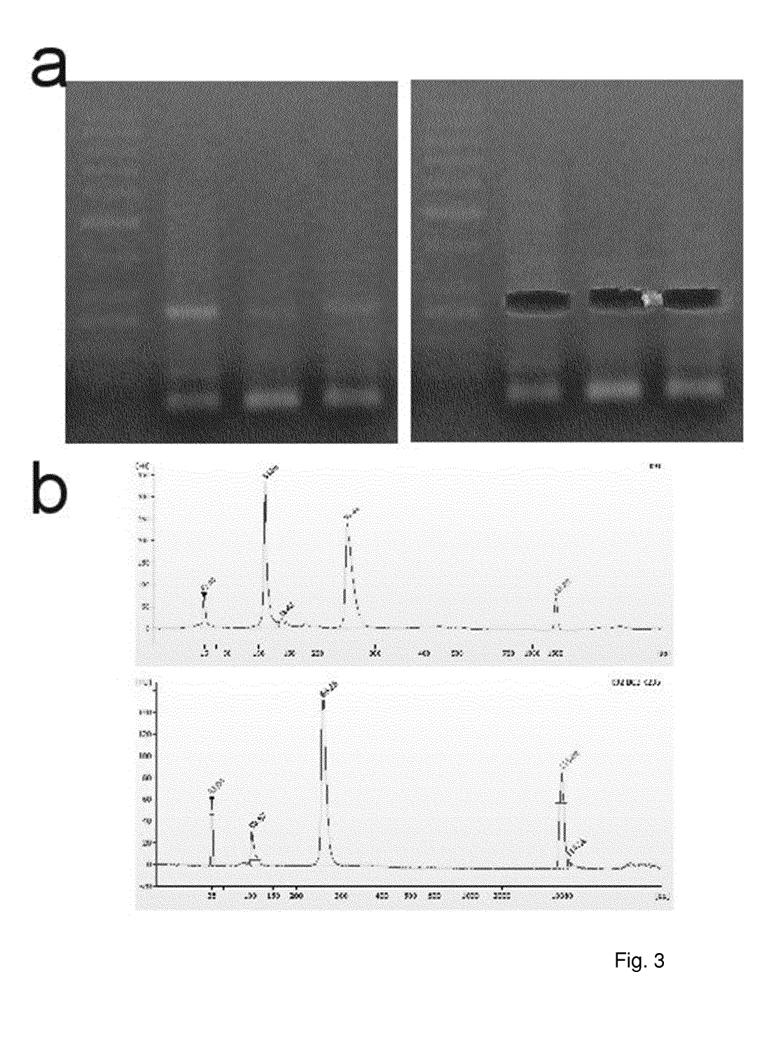

FIG. 3 shows the purification of the PCR products. (a) PCR products were separated on agarose gels and purified. (b) Bioanalyzer Profile shows significant accumulation of the correct PCR product.

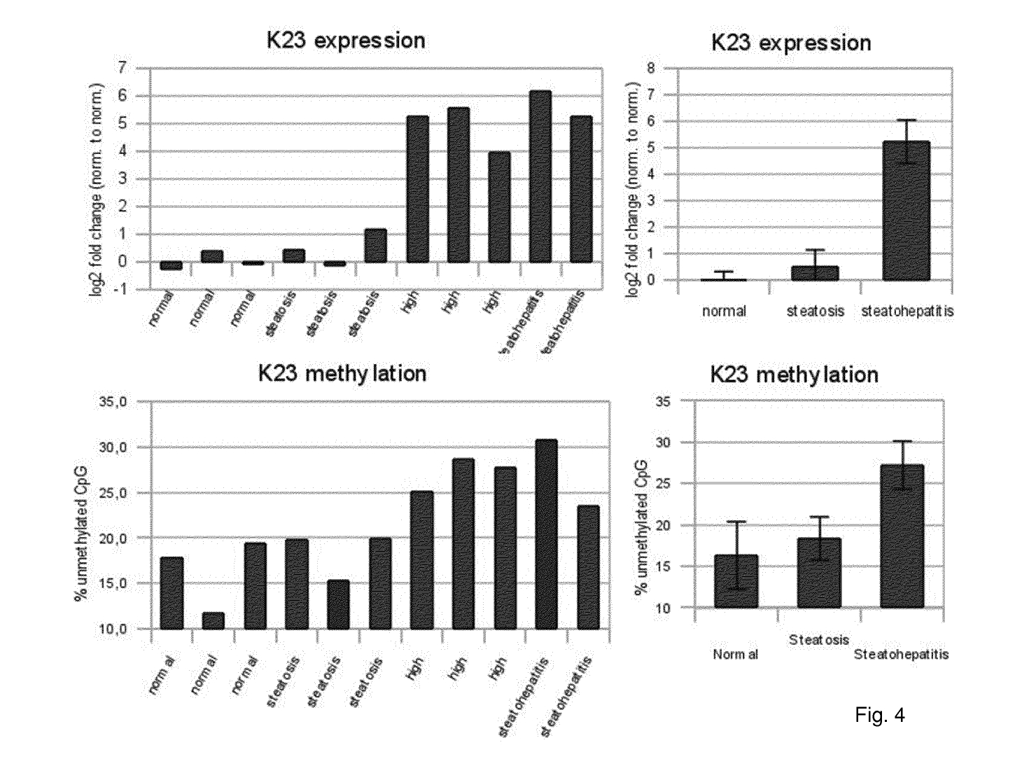

FIG. 4 shows the correlation of KRT23 expression and KRT23 locus demethylation in liver. KRT23 expression and demethylation correlate and are highest in steatohepatitis.

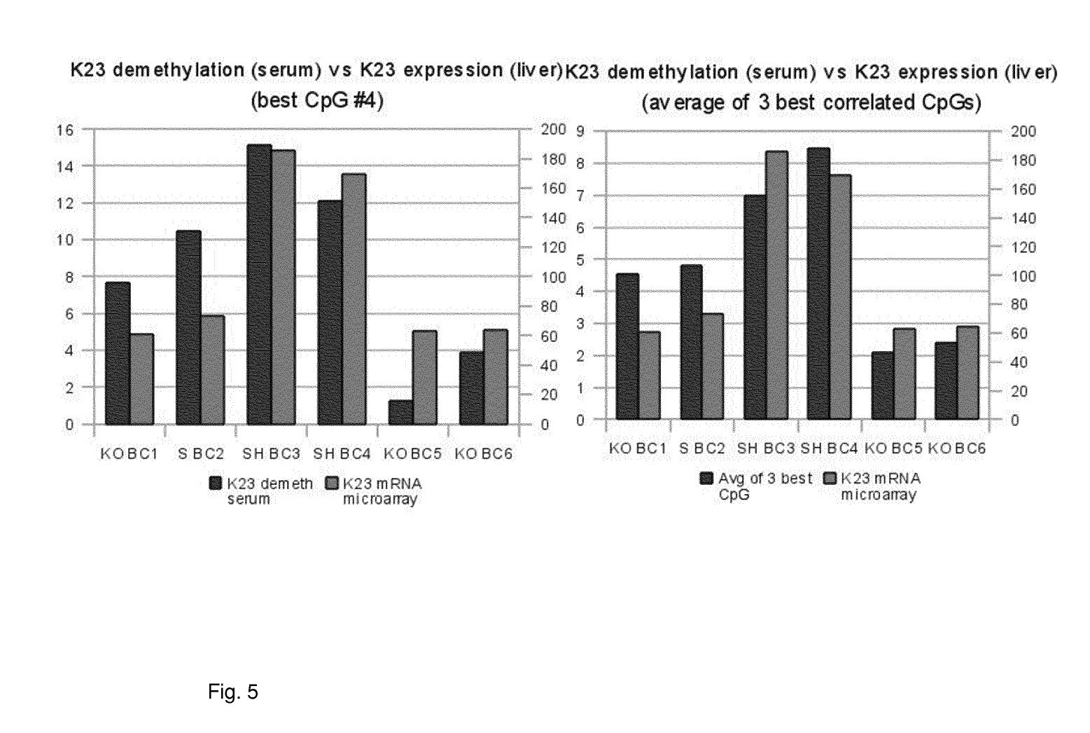

FIG. 5 shows the correlation of KRT23 demethylation with the pathological phenotype and KRT23 expression in liver. Evaluation of specific CpG sites allows an exact correlation of the pathological phenotype with demethylation of the KRT23 locus (KO: Control; S: Steatosis; SH: Steatohepatitis).

### EXAMPLES

In the co-pending application EP 11 195 537.3 (and the PCT application based on this priority) it has been shown that expression of KRT23 is a marker suitable for differentiating between steatosis and steatohepatitis. Expression of KRT23 is activated in steatohepatitis only; in liver tissue which only shows steatotic changes, no expression can be detected. Measurement of KRT23 expression is based on the amount of m-RNA or protein in liver tissue or body fluids.

According to the present invention, such differential diagnosis can be achieved by determination of the methylation status of the KRT23 gene in samples containing DNA, for example blood derived samples containing free circulating DNA.

Gene expression is often regulated by methylation of DNA upstream of the start of genes or in the first exons of genes (Felsenfeld et. al. Nature 1982, PMID 7070505). This DNA methylation can be measured with several methods, for example by methylation specific quantitative PCR (Zhang et. al., Int. J. Cancer, 2008, PMID 18546260), by sequencing bisulfite treated DNA (Frommer et. al., PNAS 1992, PMID 1542678) or by extraction of methylated DNA by antibody pulldown followed by high-throughput sequencing (Weber et. al, Nat. Genet 2005, PMID 16007088).

Patient blood contains small amounts of free DNA that is shed into the bloodstream by tumors or other tissues. This free, circulating DNA can be investigated for relevant biomarkers, like the changes in methylation pattern described here.

### Task:

Development of an assay to monitor progression of steatosis to steatohepatitis by measuring the methylation pattern of KRT23 in the free, circulating serum DNA.

### Methods:

Extraction of Free DNA from Patient Plasma

Free DNA was extracted from patient serum with the QIAamp Circulating Nucleic Acid Kit, Qiagen (Qiagen, Hilden, Germany). All serum samples used in the project were collected from patients after full consent and under a valid license from the local ethical committee. 5 ml whole blood were collected in EDTA supplemented tubes. After centrifugation the plasma fraction was separated and stored at −80° C. 1 ml aliquots were thawed and used for analysis. Usually approximately 20 to 40 ng of free, circulating DNA could be extracted from 1 ml of plasma (FIG. 1)

### Conversion of Methylated Cytosine (Bisulfite Treatment)

The whole amount of DNA extracted from 1 ml of patient plasma was converted using the EZ DNA Methylation Kit (Zymo Research, USA). This treatment converts all unmethylated cytosines into Uracil. In the course of this conversion 90% of DNA is degraded and the remaining DNA is heavily fragmented.

Amplification of CpG Methylation Sites from the First Exon of KRT23 by PCR

The region 6 kb upstream of KRT23 and its full coding sequence was screened for sites of methylation in the UCSC genome browser. The first exon of KRT23 contains multiple methylation sites in the chr17:39,092,678-39,093,032 region. After identification of this target region primers which amplify this region and the included CpG islands (FIG. 2) were designed using perlprimer software (PerlPrimer v1.1.21, Copyright© 2003-2011 Owen Marshall). The primers had the following sequence:

(Seq.Id.No. 3)
KRT23-fwd: GTGGTGAAGGATAGGGAGAT
(Seq.Id.No. 4)
KRT23-rev: CCAAAAAATAAAACAAAACTCAAC

The target sequence could be amplified from bisulfite treated DNA with this primer pair.

### Purification of PCR Products

Extraction of DNA from liver tissue yielded enough DNA to amplify a specific PCR product without contaminating bands. Due to the small amounts of DNA extracted from plasma samples the PCR reaction amplifying the target sequence was suboptimal and yielded a small band of primer dimers in addition to the desired product. These small fragments need to be removed before sequencing the target fragment. DNA fragments were separated on 2% agarose gels, the target fragments were excised from the gel and DNA was extracted from the agarose matrix using the QIAquick Gel Extraction Kit (Qiagen, Hilden, Deutschland). Final quality control using the Agilent Bioanalyzer (Agilent Technologies, USA) revealed enrichment of the desired PCR product (FIG. 3).

Sequencing of the PCR product using Ion Torrent Personal Genome Machine (PGM)

Purified PCR products were bound to beads and amplified in an emulsion PCR using the Ion Torrent OneTouch system. After PCR reaction the template bearing beads were enriched and loaded onto Semiconductor Chips which were then introduced into the Ion Torrent PGM and the sequencing reaction was started.

Next generation sequencing can produce very high numbers of reads in a single sequencing run. This allows to generate a robust statistical value of the methylation status of a single CpG in the target sequence. In the present analyses between 2000 and 6000 reads were analyzed per sample.

### Analysis of Sequencing Data

The sequence reads obtained for each sample were individually compared to the reference sequence by a combination of perl scripts and ClustalW (Larkin M. et al. Bioinformatics 2007, PMID: 17846036). After alignment the sequence of each CpG site in the target read was determined. The number of C and T reads was summarized over all reads and the percentage of thymidines at this position determined. This percentage represents the demethylation of this CpG island in the free DNA from the patient serum.

### Correlation of Liver Pathology, KRT23 Expression and Demethylation of the KRT23 Locus in Liver Tissue.

The expression level of KRT23 mRNA is different in steatosis and steatohepatitis, confirming our earlier results (Starmann et. al., PLoS One 2012, PMID 23071592). Using the assay described above a similar result can be gained from analysis of the liver DNA. DNA was extracted from liver tissue, bisulfite treated and the KRT23 locus was sequenced. The demethylation of the KRT23 locus correlates very well with the expression level of KRT23 in the same tissues (FIG. 4). This allows drawing the same pathological classification from the methylation patter of DNA as from the gene expression values of KRT23.

### Measuring KRT23 Demethylation in Patient Plasma

Measuring KRT23 expression levels from mRNA levels may necessitate a liver biopsy which is costly and implies possible complications. It is necessary to create a non-invasive assay to establish KRT23 as a biomarker of liver disease. The measurement of DNA demethylation of the KRT23 gene and promoter from free, circulating plasma DNA presents such an assay. As shown in FIG. 5 KRT23 demethylation levels of select, individual CpG sites demonstrate a high level of correlation to KRT23 expression in the liver and thus pathological classification.

### SUMMARY

Measuring demethylation of the KRT23 locus in the chr17:39092678-39093032 genomic region (or up to 6 kb upstream) in the free, circulating DNA of patient serum allows drawing conclusions on the pathology of the liver parenchyme, especially in the differentiation of blunt steatosis and steatohepatitis.

Seq. ID. No. 1:

Krt23 gene (chr17: 39,078,952-39,099,836); preferred methylation regions are given in bold; sequences used and amplified in the present examples are highlighted.

>chr17:39078952-39099836
GCTAGTGCGGAGTTTTATTGGCTACAAAATAGATGCAAAATGATGAGAATCTGAAGGCTG
CAGTAGGAAAGTAGAGCTTTACCCTCATAAACTCGCACTTTGATTAGAAAAGTGCAATAT
ATTAAGAGCATTATGAGAAGTCTGGTGAGACTGTTACAGAAAAAAAAAATAAAAGTTTCT
GAGTCTGATAATTCCAAGGGTATCTTTTAGAACTCACTCACTGGTGTCTGTGCAAGGACT
TTCCTTGGGGGAAAATAGATTTTACAACAGGCGGAAACTTTCATTGGTCTCATGCGTGCT
TTTGGATTTCATTCACTTGACAAAGAACTAATCTTCCGTTGATGGTCTCCTGGGTTATGG
CCTTGATCTTTGGAGTTGCAGACACTGAGAAAAAGAGCATGGAAGATGTCACTGCCATGC
TTCTTCCACCATCTGAACTGTACTCATTTTCATTTCTCTGCTGTCAGCCCCTGAAGGCTC
TCAGTGCCTTCCAAATGATCACTAAGCAATGAGGTGGGCTCTGCTGCTGGGAAAAGGGAT
TTCCCAAAGGAGGGATTTTCTCCCACTCTCCAGGAAATGAGGATCTCTGAACAGTGTCAC
CTTCTGGAGGTGAAGACCTTCATCATAGGTGTGCCTCATATGGAACCTAGTCTCTGACCA
CAAGCACTTGAGGAGTTCTTTCTCTGCCTGTGGGCTTTGTAGCACTGCACAGATTCCTTG
AGCTTACAGAGCACTTAGTGCTATTCAGGGTACCTCATTTTAAAGTCTAGAAAGCATGCC
ACATGAGGAATGGGTGAGAAGCCAAGGATGCTTACCATGGCTGTTTTCAAATATATAACT
TATTTGAATAAGTGCCTGTTGAAACATCAATAAAGCAATTCTAGACCATAGGGAAGAATT
GAAATCATTAATAAATTTACAAGGGAGAATTTTGTTAAATTAAAACCTCTCCATTGGGAC
CATCCCATCAAGTATGGGTTCCCCATCCTAAGACATGTGTGAAGAGAGTCTCAAAGACCC
TTGTCTTATTTTGTAGATGGGTAGCAGTGAGACCAGCTGTCCTGTGATGATTACTCCAAC
TGATTCTTTGAATCTCCAATGCCTTAGAATAGGTTTATCACAAACTAATCAGAAAGTGCC
ACTTGTCCCAGACTACAGTCTCCTGCAACTTTTCTGTGAGATATTAAAGGTAATCTGAGA
AAGCATGTTTCCAGGTCAAAGATGTTTGGGAATACTGGGCTAAACAAAGTTAAACTTTTG
TTGTTTCTACCTCTTTCTTGTTCCTTTTCTCCTTCAAAGCAGTACCTGTTCCATCTTTAA
TACACTGAACTGCAACAGGACTGTCCAAGAAGAGGATCATGGCACACAGTATTTCTAACC
TTATCTGGCCATAGAACACAGTGTTCGTAATACACTATTCAAATCTCATGGAACACGAGT
GTTCAACAGAATCATCTGAGAAATGAAATCATGAAATCATTGCCTCTTAGTTTTCTAGGG
GTTTAGTAAACTGACTCCATTACTTAATAGAGACATAATACTATTAATTATCATTTTAAT
TTTCTTTACTGCTTGCTTTTACCTATATTGAGGACAATAATGAGAGCTTACATCATTTTG
TATTAGCTAATTTGTTCATTGAAGATTCCTGACCCAGGACATTTTGAACCCAGGACATTT
GGGAGTACATTAAGGATTATGACTACTACAAAATGGATAACCATGCTAATTCCTTAAAAA
CAGAGATTCAAACAATGAAGGAAATTTTTTACCTTTCATGCTCGACTTTGATTCTTCCCG
TGTCCTATAAAAGTGATAAAGGTTAAAGCTCACTTTAGTATTAGCAACAATCATAACAAC
TTCTCAGGATGAACTAACTTCCTTCGCTTGCTTGTAAATCCTCTGCTCTTTAATGAGGAT
AATTGTCTGAGCTTAGTAGTCTCAAGGTCATGGGTTCACGGGAGAACCCGTCTGGATACA
GAAGGCATTGGGTACCGCCTCCTGCTAACCCTGGAGTCAGGGGAAGTCCACCAACCATCC
CCTCCTTTCAGCTGCAAGCTTGGTTTTCTCTAGGATCCCTGCCAGGGTCTGGGTCTCAAG
CCTGAGACCCCTGAGCTTCTCAGGCTGTTCAAAGTTTGGTCTAGTGATGTTTTATTTATG
TATTTTTATAAAACTTCTATCCCTTCCTTTCGGATCATAGAATATATATGCCCTTTATAT
GGCCAGCTGTTGTTATTTCCTGGGGTGGCCAGAAATATCAGATTGAAAGTCTAGACATCT
CTAGCTCATATGCATCTTTTTTTTTTTTTCTCTTTTGCTGGTGGACTAACACATTCCCCC
TGCCTTCTTTTTTTTGGAGCCAAAATTGTGTGCATTCCTACTGGGAAACACAGTGGCCAA
ATCCTTTTGAATTGTTTCCTTCTAGAGACTTTAACTCTTCTGACTGCAAATCTTAGTGTC
CTGTGAGTATTAGTTGATTAATTATACTTGCTGCTTAGTGAAATACAGCCAGCTATAGGT
ATCTTCTGGAGTAGCTCAACACAACTTTTCTCTTGCTAGAGTGACTCTTGCTAACAGAAC
CCAAAGATGCACACATATACCCACAGGAGCTGGAGGTCCCTCGCATGCTCCTCTCGTGCC
AGCCTTTGCCTTACCCTTCACTCTCTCCCTCCAGGAGCCGTCGGTACGTGGTGATTTCCT
TCTCCAGGTGGGTTTTGATGCCCAGCAGCACTTGGTATTCATTGTTCTGCCGCTCCAGTT
CATGGCGTAGCTGCGTCAGTTCCTCCTCATAGTGGGAGATGATCTCTTGCATGTCCTGGA
GCTTGCAGGAGTACCGAGACTGGGTCTCGGATAACATGTTTTCCAAAGCAGATTTCTGAA
AGAGGAAATGATCTTCTGTTAATTAACTGATGGCTTGATTGAGTCAATAACAGGTGATTG
TTGAGTACTTAGCAGATGTCGACCAGTTGGCTAGGTGTCTTGGTAGCTGCTGAGGACACC
AAGATGAGAAGTAGGCTGACTTACAGGTTTGGGACTTGGGCAGTCAGGAGTTGAGCAGTG
TTAGGACTTTTGAAGTCCATTGCCTTGCTCTGAACTGTTGTTTAACTCCTTTCATTTTAT
CTTTGACCTGATTGAAAACTCCCAAAGAGCGACAGCAACTGGGACTGCTATCTCTTTGTA
TCTTCGGAGAAGGGTCTCAATTAGGGATGCCAGATAAAATAGAGAACATCTAGTTAAATT
TGAATTTCAGATGAGTAATGAGTAATTTTTTAGTGTAACTATGTCCAAAATATTGCTCCA
GTATTGCATGGGACATGCAAAAAAAATTGCTTTTTATTTGAAATTCCAACTAAACTGAGT
ATTCTATATTTTAATTTGGTAAATCTGGCAACCCTAGTCTCAAGCAATGCTTTTTGATTA
TGACGATGGTAAAAAAAAAAAATGTCATTTTTTGAGGAAATTCTAACAGCTGCCAAATGA
AGTTTCTGGGCTAGAATATGGAGGATTGAGTTCAAGTCCCAGTTCTGTTTGCATGTAACA
CCTGCTTTTCTGAAGCGCATAATTTTAACCTTAAAGAGAATCTCCTAACTGTGCTGGATA
CTCTCCCTTTATCGTGGCTGCTAATTTTCAGGGGGCCGTTCATGCTGCCTGGCGGTTAAA
GCACATTTCCCTGGTCAGCCAGTCTCCTACTCTCCCAGGAGGTTATTTTGTTCCCTCTCC
TCTGTCCTTGCCATCCTTCCTCGGAGTCCATGGCCTCGCTTCCTAATTTTGTTGATGAAA
TGAAAGCAATCGGGAGAGACCGCCCACTGCTCCCTACATTGCTCCCAGGCACATACTTGA
ATCTGCATCTGGGTGCTGGCTTCCCTCTGTTCTTTTGTGTGAGCTGTCTGTGCTCCAGTC
ATCTCGGCAGTTGCCCGCCTTCTTGCGTTATCAATATTCTCTCTGGATTATTCCCACCAG
CTTTCAAATAGAATAATTTTTCCTACCTTAAAATATATCTTTCTTAAACCCATATCTACC
TTCAGCTATCATCCAATTTCTCTGCTCTCCTTCAGCTACAGCAAAGCTCCTCAAAGAGTT
GTCTATGCCTGCTGTTCCTCTTGAGACCCCTCCAAGGTAGCCTACAAAGCCCTACACTGT
CTGGTTCTGTTGGGACTCCGCGGCCTCACCCTGTCTTCCTCCCTCTTCCCCTCCCTTCCC
CTCCTCTTCTCTCCTTTCTTCTTCCCTTCATTCACTCACTGTAGCCACATGGACTTCCCT
GCTGCTCCTCCAACTCATCAGACACACTTGTGCCTTGTCGCTTTTGCACTTGCTGTTCCC
TCCTCTTGGAACACATCCCATCACCACATCAAGCACCTCCTTACCTCTTTGCTCACTGCC
ACTCTAACAGGAAAACCTTTCCTGACCACCTTGTTTAAGATAGCAGCTCTTCACCCTGGT
GCATCTGCCCTCCTCCTCTGCTTCATCTCTGTAGCAATTTTCTCCATCTAACATATATTT
TGATGATTTGCACATTTATTATCTGTCACTCCTACTAGAATATAAACTCCTTCATTAAAA
AATCCTCAGTGCTAAGGATTGGTGTCAGATAGTAGAATGCATGAAAATGGGTATACATGC
CTCGCCTGTTTCACAGGGCCATTGTGAAATACTGTATGTTAAAACGTTTTGAAAGTTGCA
CTATAATTATTTTTGTACTCATGATCTCTACGTGCACATTTGCAGTGTCTGTTCACCATC
ACGGATGCATAAAACACAGCAGTCATTTACCCAAGTGCACCTGCAACATAAATGAACAAA
TCATTGCCTCTCAAGACTGAGGGTAGAATCGCACTTGGCAATTATATGGATGAATGATTA
GAAGACTCAATAGGGTATACTGGGAATCAAATGATAGACCTTCATAAATTTTGAATGCTT
ATGACCATGCTAAGCATTTTTCTAAACTGTGAAGATGATAGAAATGACTTGGACACAAGA
ACTTAGAGTTTTCTGGGTGATAAGACATATACATAAATGAATAGGGCAGTGCTTTGGAGT
TAGCCAGGCCTGGATCTGCCACCTACCATGTTGGCACTGGAATTTCTCTACATTGGGGTT
TGAGGGGGGTAGCCTGGTTGTAAGTGGAGAGGGCACTGATAGAAATTATGGGGGTCTAGA
ATCCCCCTTCCTCCAAATAAATCATATGGTTTCATCACCAGTTACTTACTAGCTGTGTGA
CCTTGGCCAATTTATTTTACTGTTGGAGTTTTGGTTTCCTCATCTCTAAAATGGGGACAC
TAAGACCTATCTGGGAAGGTTACTGTAAGGATTAATGACATTTAATATCATGAACTACTT
AGCACAATGCCTGGCACCATCGGGACACTCAATACATGGTAGCCCTTTATTATGGTTCTC
ATCATAAACAATTTGGATGGAAAGTGGAAAGTGAGAGTTGTCCCAAGGGAGTTGAGGGTC
AGGGCCTGGGAGAGAGGTCTCAGAAGGCTTCGTGAAGGAGGTGGTGTGGGATTCACGGTT
TTCCTAGCCTTGACTCACCGTGCTGTACTGTGTCTGCAGGTCAATCTCCAGGGCCTGGAA
TGTGCGCTTCAGTTCGTGGATGTCACCTTGTCTGCTCTGCACAGTGGCTGGACTGGCTGC
CTCCTGGGACATGGCTGCAGACTGTGGGACCAAGCAAGGCAAAGGCGTCAGCATTGGGAC
CTCATGGAAATGCAACCTTTGGGAAGTTTGTGCATCTTTCTTTTACCTGTTCTTTATACC
AAGTGTCCAAGTCTCGATGCTTCTTCTTTATTATAAGCTCATATTCTTGTCTCATATCCT
CCAGGACCTTAATCAGATCTTCCCTGGGACCTGTATCCACCTTCACATTGACATTGAAGT
CACTTGGCACATGATGCTTCTCCATTTCCTATTTAATAACAGAGAAAGGAAATGAACCGG
CTTTGACAATCAGAAGGCTGGATTGCCATGTTGATTGGCAGTGGTAAATAGATCTTTTAA
ACATTCCGATTATGTGGTTGCTCCCACTGTCACTGGTTTGGTTTCTCAAATATTACTTAC
TTGTTGGTGATAAAACATTGTATACTTTTTTCTCTAATAATTTTATAAAATAAAGAATAT
CTTTGAGACCAACCTGACCAGCATGGAGAAACCCTGTCTCTACTAAAAATACAAAAAATT
AACTGGGTGTGGTTGTACACACCTTTAATCCCAGCTACTTGGGAGGCTGAGGCAGGAGAA
TCGCTTGAACCTGCGAGGTGGAGGCTGCAGTGAGCCGAGATTGCGCCACTGCACTCCAGC
CTGGGTGACAGAGTGAGACCCTGTCTCCAAAAAAAAAAAAAAAAAAAAAAAGAATATCAT
AAGGCAGGTAATCAAATAGCCTCCATGATTACTGACTTGATCCGTGGTTGAAAATATTTC
AGAAAGAATTATGCTTCCATAGTGTGCAAAGTCCTGTTCAGCAGATGTGGATTGGTGAAG
TGACAACATAGTTAAGCAGCTGAACCTTACAGGCTATAGGCCAAAGCTTTTGGGAAGCAG
TTCAAGAGGCAAACAGCAGAAAGAAGGTGTAGAGGCTTTCAAAATCTCATCAATGCCTTG
TGACACTGTAGGGCTGACGCCCATAGGATTTCATGAAAACCAAGCAGCCACAGGAAAGGT
TTAATTAACAATCATTGTCTAACCAGCTATTCAGAAACTGAGCTCTTTGATGTTGAGAAG
CAATTTTGTTTTCCTAAAATGGGAACTGTTTTTCTATCATTTTATCAGAGTCTTGAGGAC
TTGTGATGGATGGTAACTCTAAAAGTCAGTTAAATGCTTCTGGAAATTAGGATTCTTTAT
TTTTGCCCCACATCACACTTATCTTGGTTGGTAGCAGACTCTCTTCATTATATTGTCCCC
TAACTTGCTGAACATTGGGCCAGTGATGGAAGTCCAGTCTCACGCATTGATTCTTATAGT
AACATTAACAAGCACATCTAGGTAAACATTGTGTATTAACAAAACTGTTGACCAATTCAT
CATTAATTATGATTGCATTTTGGTTGTTGTATTTGACATGTGACTAGCTAACCTTGAAAT
TAGAAACATTAGATATTTGTATGTTTTTCTAAAGACTATTTAGGGTGAAGGACAAGGGGA
TTGGCATCCAGGTGGTGGTTTCTTCTGTAGCTGTCGAATCAGCCTGAGATGTGCAGGCTA
AGAGCTGTGCTTATTAGCAGGTGTTCCTGGGGAGTTGTACCTGCTCATGGTGCTTCTTCA
TGAGAATGAGCTCTTTCCTCATTCCTTCCACCTCCTGTTCTAGGTCTGTTGTGACAATGG
TCAGGTTGTCTAAGGTCCTTCGGAGGCCCTCGACTTCAATTTCCAAGTCTTTCTTAAAGG
AGTGTTCATTTTCATACCTTTGGTGAGAAGGAAGAGAAGAGTGCACTCATTGTTGGAAGG
CCATGGTTTTTGAAAGGATTCATTTTCATTTAGAGGTGAATCTTTTACTTTTTAGGTTTA
AAGAGAGCAAATGAATACATATAGGATGTCTTTTTTATGCCAGTTTCATGCTACACTTCT
CCATGATGGTGACTCAATGTCAACTTTTAAATTGAGTAGGGAGAATAGGATAATAAACAG
TCATATACCCATTATCTATATTTAGCAACCATGAATGTTTTGCCATATTTGCTTCATCTA
TTTCATTGCTGAAGTATTTTAAAGCAGATTATAGATGTGATGACATTTTAAGACTAAATA
CTTCAGGATGCATCTTTTAAGAAAAATATATTTTAAAATAAACAGGCCGGGCACGGTGGC
TCACACCTGTAATCCCAGTACTTTGGGAGGCTGAGGCGGGCGGATCACGAGGCCAAGAGA
TTGAGACCATCCTGGCCAACATGGTGAAACCCTGTTGCTACTAAAAATGCAAAAATTAGC
TGGGCATGGTGGTGTGCACCTATAGTCCCAGCTACTTGGGAGGCTGAGGCAGGAGAATCG
CTTGAACCTGGAAGGCAGAGGTTGCAGTGAGCCGAGATCACGCCACTACACTACAGCCTG
GCAACAGAGCAAGACTCCGTCTCAAAAATAAACAAATAAATAAATAAATAAATAAATAAA
TAAAATAAACATAACACTACACCTAAACATTAATAACAACCCTTTAATATCATCTAATTC
TTGATTCATATTCAAACTTCCCCATACTATAATGTGCTTTATATCTGATTTGTTTAAACT
AAGGTTCATTCAAGTAACACACAGTGCATTTGCCGTTGCTATGCCTTTTCATTATAAGAT
TTTAGATGCGGGTAATGGCAAGATGAGTTTTCCTTTTTTTTTTTTTTTTTGAGATGGAGT
CTCACTCTGTTGCCAGGCTGGAGTGTTAGTGGCGTGATCTTGGCTCACTGCAACCTCCAC
CTCCTGGGTTCAAGTGATTCTCCTGCCTCAGCCTCCCGAGTAGCTGGGACTACAGGCGTG
TGCCACCATGCCCAGCTAATTTTTGTATTTTTAGTAGAGATGGGGTTTCACCATGTTGGC
CAGGATGGTCTCGATCTCTTGACCTCGTGATCCGCCCGCCTTGGCCTCCCAAAGTGCTGG
GATTACAGGTGTGAGCCACCGCGCCCCGCCGAGTTTTCCTTTTGTAAAGCAAACAAACAA
AAACCCGAACAACCCTGTGAAGAAGGAACTTACTTGAGGTTGAAGTCATCCACTGCCATC
CTGGCATTGTCAATGAGAAGAATAATCTGAGCATTGGTCATCTTACCATCCACTATCTGT
AAAACATGCACAAAAGCACAAAAGGTTATCTCACTTGGACACACCCACACATGACCGCTG
CATGTCTGTCCTCTAGCTTCCTGAATATTTGACTTGTTAGTACAGACAACTTCTTGTTCT
GGCAGTGGAACAATGAATAACCGAATCAGTAAATGCTTAGTGAGCACATACTAGGTGCAA
AGCAGAATGATTTTAAAATAAGAATATGATCTGCAGTCAGTGTTACTGCCTTTTTTCTTT
TTATGTTGAGTTTTGACAGGCTTGGATTGATGCAACTGCTGAAAGAATGATATAGGAAGG
GGAAGGACAGTAATTGTTCAAAATGCCCTCTAACTTTTTAGGTTATGGACATGTCATAGC
ATTTTGCTTAATTAAGCTCATCCACAGCTGAAGATGTTAAAAGGACTTTAGGTTCTGATC
CTTTTCTTATTGTATTAGGTTGGTGAAAAAGTGATTTTTGCCATTACTTTTAATTAGTTC
AGCCCTCCAAAAATACAAAAATTATTATTTCAGGTGAACATAATTCTTACTTGTGATAGC
AATCATGTTTCTAATCAGTGTTACTGAGCAACGCTAACACTAATATTCAATGTATTTTGT
AATAGTCATCACAGAACACATTTTTTTTTTGCCCGAAGTGACTGTAACTTGAAGGTGTGT
TTATGTCATTGCAACTTATTCTGTGATGAGAATGTGCTCATTGTAGCTATTAAAATATTA
ACTGAAATAATTCCTTACCATCTAAGGAAAATGCTTAAGAAGCTTCAGTAGTGGTAAAGC
CTCCAGTTAAAGACTTTCACTAGATGTTCGTATAATTTTTTTTTTACTTTGAAAAGAAAA
ATAGTATTGAAAGCACTTACTCGAGCAAGTGTGTATACATTTATGATTTGTAAACACTTG
TAGGTATGTTATAGTCAGAGAAATAGCACACATTTGATGTATGTCACTGTCTTTAGGGTA
GAAGATGTCTCGTTGTCAAAAGTGTAATAATGCTTTTACGCCAAGTGTTGTGCTGGACTA
GAAACTTAGAGCTTTTCTTTGGTTCTGGCTCTGTTCTTGACCATCCATGTGGATGCATCA
GGCTGCATCACTCTACTCTCCAGGCCTCAGTTTCATCCATTGTCAAATAAGGAGTCTGTA
CTAGAGACAGAGGACCTTAGAGGTCTTAGCTCTAAGTTTCTGTAATTTAGTTGGAGTCAG
AGAATCCTGGGTAATTCCCATTATATTTACATAGCTCATTCCTTTATTTAGAAAGCATTT
TCTTAGGACAAAGCTATACTTCAGTACAAAGGCACCTTTTAAAAGTCTGGTCCTATTTAA
AGCTCCACTGATCCCATGGAATTTGGTATCATGAATGCACAATAGCAGGATTTAGAGATG
GCATGAATGCTGGGTCTGGAACTCAAAGAGCAGGGCCTGGGATCCTGCTCCGCGCTTACT
GGCTGGGTGAGATTGGCTGAGTGAGCTTGGCTGAGTCTCAGTTTATTCATCCATAAAATA
GCAATAACAATAGTTACTTCATCAAAGAGTTATGAGAATTAGAAGAGAGTATGTCAAATA
ATGTCAAATAGCATATTATCGGCATATTATAGACAGTGAAAAATTGGTCGAAACTGAATT
TGTTCAGAAAAAAGTAAATAATACACCTTTCTTTTTAAAAATCTAGCACTTTTATAGATT
CAGTGACTACACTGTGAACATCCCCAGGTTCTATATGCTGAGATGCCTGCTCAGACTTTT
GCTGTTCATGGTGGGACATAGCCAGGCTGGCTGCTCCTGCCTCATTCTTTTTCCTCTTGT
GGGATTCCAAATGGTACTATGGCCCCTTCCCTTGCTATAAAGAAGGACACTTTTAGATAT
TTCCTTGTCTCCTCTCCACAGTGCTGCTTAGGGGCATTCTTACCAATACTTCTCCCTCCA
GACCCCTTATTCTTTGCATTATGTAAGCTCCTGCAAAATGAAAAGTAGCTCTATCCCAAA
GAGCCTGGGGGCCATTGTGGGGTAACCCTTACTTACTTGTGCACTGAGGTTTTCTATTTG
ACACACACTATGAGGTGTTAATCTACATCCAGAGAATCAGAAAATCAAAAATACTGTCTA
TGGGCTTATTTAGCTTTTCTGGGTTAAATTGGTAATTTATCCATTGCAAAGTCCACCTCA
AGTGTCTGATTCCCCCAGCTATCATTCTCATAGCACACAAATGTGAATTCTGAAATGTGC
TTGATATAGAGCACGTTTAAAACTATTTCCCATTATTCAGGCTTACTTAGTACAGAATTA
TGATATAACCATCATTTCTGACACAATCTTCATGGTAATAAGTGGATGAAGATGGGTTGA
GAAACGATATATGAGGCATAGGTTTTTTACTGAGTGTTGTAATTAATCACCCAGATTCTC
CTGAAGGCCCTCAGTTCATTCTTTTAAAAGCAAGTTGGCATATTCTTGCAGCATTGTTGC
TTCAGGATGTTTGATCTACTAGGGAGAGGAAAACAGAATGTTTGACTGTAAACAAAAATG
CCAAATATAGTCCGATTGAGAATCATTCAAGGTGTTTTATTTATGATACTCCTATGACTG
TGTCCAATTCTGGATTTCAAAAGGATTGAATTAATTTTTAAGCTCAACTTGTTATCAACC
TTTTGAGTGATATTTAATATACCTAAAGTGAGGGTACGGGAGAACTGTATCCACTCAGAG
CATTTCACCAAGGTCCAGGAAGGATAAGCCCCAGGTAATTGTGGGACCTCCTTGCACCTC
TGTTTTCTCATCTTCAAATGAGGGAGGTTGGGGTAGATAGACAACCTCAACTTTAAAGAG
TTTCTTTCTTCAAGTGTAGAGAAAACACAATGTTAGAGGTAAAAATCCAGAAAAGAGTGA
AATTCAAAGCCAAAGAAGTTTCCTATTCAATCTCAGAACCCAGAATTCCAGGTCATCTGA
AGGGTGGGCAGGTTGCTCCCCACACCCCCCATTGCACCTACTCACTTTTCTCAATTCTGT
TCCAAAGGATTTAGACAATGATTAGGTCTCAGTCTCCTTATCTGTAAGTTGAGAATACAA
ATACCTGCTCCACACAGTTGTGAGGATTAAATGAGACAATGTGTATGAAGTATGTGATGT
GTGGTTAATGATGCAAGTATAATGCATTATCTTTATTCATACTAAGTGTCATTTTTATAT
GAGGTGGGCTAGGCAGCACTACACTGTAGTGAAATTTATGAGGCTACTGGAAACAGACCT
GAGTTAGAATCCCACCTCTGCTTCTTGCTGATGGCTTTGGATGGCTATTTCACTTTTTTC
CAAGCTTCCATTTCCTTACAGGGTTCTTGCAGAATCAAATAAAATAATATGGAACATTTC
CATCATTGCAGAAAACTCTTGGATAGTGTTGACATGTGTATTAAATATCCAGCAGGATGC
CTAGGTACATAATAAGTGCTCAGTAAACATTTCCCTCTCTTTCCTCCCTTTCTTTTTTTT
AAATCTCTTCCGCGAGCTCATAAATTATTTGACTGGTTCTAACCTTGTATTTCTTTTGGG
AGTCATGGTCTCCCTTTGCTCTCCATTTGCAAAAATCTTCAATTCCTCCTTTCTACAACC
AAAGCAACTTTATTAAACAAAAAAACATGATGAGGACTGTTGAGTCACCTTGTTTTTTTT
TTTTTTTCATAAAGACAACCATGCACTATATAGTTAAGGTAATTACAGTCAGTTATTAGG
TTGATTATTCAATAATTAGGGTTCATTACAAGGACAGATTTATATTATCTTGACTGGTCC
TTCCTACATTCAAGCAAGGATGACTGAGCATTGTGAAATAATATATATAAATATTGGAAA
CTTGTGATACCCTTTCTAGGGGAGGTTAAATCATGGTCTTACAGGGAATTTTCTGGGACT
GCTTTATATAAGAGGGGAGTTAGATAGCTTTAAAACATAACCTATGAGATGATTGGATTC
CTTCTACTTCTAGCTAATGTTATGAGACATCCCAGAAGGTATTTGCTTAGCTTTTTTGAG
GTTTGGTTAAAGAGTTCGGTTTTTTTTGAACAAGGTCTTTATAGCCATCTCAACTCTCAG
ATTCTAAAAATTATGCTCATTTTATTATTATTGGTTCTAGCAATATGCCATTGTCAGAAC
TGTGAGCAACTTTACTGCAAGCAACCCACAAACGGAAATATCTAGGGATGTCATGATGAC
CTATATGCTGGTGTGGTGACAACTTTTAGGCATAATCTTCAAAATAGTGTAACAGCCTTT
TGTCGATGGATAACCACATTGTTCAAAAAAGTTTGAGGTGGAGCAGAACTGAGGTAATGA
GAAAGTAAACACTCTCTTATGCCTGGTGCACTGAGCATGCAGCATGAACACCAGCCCTTA
TCATTTCTATGACATCGTTGGGTCATAAGTGACATTAAACAGCAGCTGGTTGCCTCACAT
TTTAAAGATGTTTTTGGTCTGTGAGAAATCAGTAGTCTCATTTTTGAAACAGTCTGTAGC
ACTTCTATCATTTTTGCTGCCTTCTTTTTCTACATTATCCATGGACAGTAGACTAATTAA
ACAAAACAGACGTTTCCCACTCCAAATTTAAGATATAATAAGAAAACATCCCCCAAAACC
TTTGTGCACAATTACTTCAAGCGCTGATGCCATTTTCCTGGAAATTGTCTTCTGGACTCA
TTTTTCCTCCCGAGGGTCCCCTGGCAAAGCAGCCCCATCTGGATCTCCAGGGTCACCCAG
CATCTTACCTGCTCCTGCAGGTGTGTGATGTTTTCCTCATACTGGGAATAATCTTTCTTA
CTGCCAGGATCTCTCTGCTGGTGCCATTTCAGGATGCGGCTTTCCAGCTTCATGTTGGCC
TCCTCCAGGGCGCGAACCTTCTCCAGGTAGGAGGCCAGGCGGTCGTTGAGATTCTGCATG
GTGGCCTTCCCATTTCCGCCTAGTAGGGGGCTGCTTCTTCCAGAACCCCAAGACCCTCCA

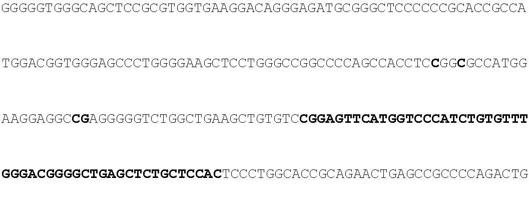

CCCTGGATGGTTTTATGGCCTTTGCTGTGGGAGTTCCCTTCTCTGACAATTGTACCAACA
AGATTGTATCATTGTGCAACTTGTGTTTCCTCTTGGTCAATCCCAAAGTGCCCCTGGGCC
TTCGCACACTGTTGTTGTTTAGAGCCACATCAATATGACAGTTTGCTTCCTCCCTTCCCC
TGGTAACCCGGAGGTGTGGCCCACGACATCAGGCGGGTATTGGGCTCAGGTATCTTTCTA
ATCTTGCCGTGAAGTTTTTCCATTGTTCATTTACCTGGAATGGGTTAGGTTAAAGTCCAC
CCAAAAGAAAGCATATTCGATGTAGTATAGATTTCCTTAAAAAAAACCAAAAACCAAAAC
CAAAACAAAACAACAACAACAGCAACAAAAAACCAGACATTAAACAACAGAGATACACAC
ACCTTTTTCCACCCCCAATAAGCCCCACCTCTAAAGGGAGGGTGAGCGAGCCTGGAGAAG
GGAAAGCCAGCTCAGAACAGGATCTCAGGTCCCAGAATAAAGCTTGCTGTGCTGAGGCAG
CCGAGAAGGTGGTGAATATCTGATGCCTTTGTGGCAGTCACTGTTTGAAGGAAAGTACTT
TCAAAGGACTCGCTCTTAAGAACAAACCCTCAATTATAGATGTAATTAATTAATGAATGA
CCGAACCTTCCACTTCCAGGGTGCCTTTCATCCCAGCACACCTCAAAACCAAACAACCCT
GGCCTGCGCTCAGGATTTCCCTTGGCCCTGGGGGAGGAATGTAGCCGTTGTTAGCAGCCC
CGGACCCCGGCTGAGCCTGCCACGGCTGGGCTCTGTGTGTCTGTGACCTGCAGAGGGACG
ATGGAAAACAGGAAACCTGCCATGGGCTGTCAAATGTGGTTGATGAATCTTATATTTCAT
CTGCCAAGGTAAAGGTTAGGAATGAATAAAGAAGCAGTCTGGGGGCGGGAGCCACGTGCT
AGTTACTGGCAGTTGGATTTAAAGGGGACTTACAAATAAAATATCAGCGCAGGAAGGGCT
TCTGGCCTTTCTGGTTCCAAGCCACTAATTGGACAGATGAGGAAAGCTGAGGCCTATGGT
GGTGGGGAGGTTAGTGGCAGAGCCAGGGCTAGATTCTTTCTCCACATAGCACTGTTATCT
TGGGTTGAATCTTGACAGGTCCTGTACCACACTTCTCTTTCATAATAATAGGACTTGCCA
ATTGGTGTAGCTCTTTTATATTCTTTATTGAATCCTCACAACTCTTTATGTATCCAGGCA
AGGGTTGTTGTCTTCATTTTGCAGACAAAGAATCAGGGTCTCATAGGGATGAATGATCCA
GAGCTGATTCGCATGTACAGTGTAATTGAGCACATGAATTCATAGGTGACACTGCCCGAG
TCTGTGCTGTCCTCCCTGCCTGGAAAGTCACTCACCCCTACCTCTTCTATAGTTCAAGGG
CTACCTCCTTCAACAATCATTTTCTGAATTCTCCTGCATGAGTTGGGTGACCCTCTAGGA
GGAAACTGCCTCTTCTGGATGCCGAGGTCCTCCAGAGGGGCAGACACCTTGGCATCCCAC
CTCATGGTGGAAGCTGGGAGCCCCTAGCCAGGCGCAGCCACCTCCTGTGCCTGGGATGAA
GTCCACTCCGTTCTTTCACTCCATTATACTGCATTTGCCACACCCTTCTGAGACCGCAGG
CTTCTTGAAAGCAGAGTTTATGTCTTCCATTTTTGAGCACCAAAGTCTGGTTTCTTGGTG
GTATTTTGCAAATGTATGTTGACTCAATCATAAACTTTTTAGAGTCAGGATTTTAACACA
GGTCTCCTGATGCCATCAACTTCTGCTCCTTTTAGGACTGTGAGTTATAGAACTAGAAAG
TAGGAAAACGCCTCTCCTGGGTATAGATTAACCATCCATGAAGGCCAATCTTTTCCAATT
ACAAATAAAGCCACCCAAAAGCACACCAGCTCTGCTCCTCCCAGTCTTACAACAAAACCT
TATCAGGGAAGAGTCTCTGAATATTTTTTATTTCACTGTGCACGAGCTGGAAAAATGAGC
CTCCAAGCCTCAGGGAAGCTTGGAGAGTGCTTAGAAAGAGTGCCGACAGTCACTTACATC
TCAGATATGAGAAAAAGACAAAAGAGACAAGTGATAAAAATAGAGCAAATGAGGGAACTA
GAGAGACTTGTTTGGCGAGTGGTGGCAACCAGTGTTCTGTGGATGTGGACGGTTCCTGGT
CATCAAAGTTATGGTCAGCTCTGGTTCCTGTTCGCCTGGACCCAGACTTACCAAGGAAGA
AAACATTTACCTTTAGCATGTGCAAGGGCCGGGCTGCACTGCCTTTTTCTAAATTCTCTC
ACCAGAATCTACAATGAAATTAATGCTTCTCTAATTATTTCAGGCATGTCTGAGTGGCAG
TGACTTATCAACCCCATAGATTTGGCCACTGTCTCATTTTATGTCCATGTGTGGGAGTTT
TGATTTATAGGACTTATCCTTGAGCAAAAGATTTCTTGTGTCATAGCAGGACCAGGGCCA
GGAAAAACTGGTGGTGAGATCTGAGAGCACTGGGGAGAAATAGTCTCTTCTAGTACTGTT
ATATGATGGTTTCCCATAACTTCTTTTTTTTTTTTTCTCAAGATGGAGTTTTGCTCTGTC
ACCCAGGCTGGAGTGCAATGGCGCAATCTTGACTCACTGCAACCTCCGCCTCCTGGGTTC
AAGCGATTCTCCTGCCTCAGCCTCCCGAGTAGCTAGGATTACAGGTGCGCACCACCATGC
CCAGCTAATTTTTTGTATTTTTAGTAGAGACGGGGTTTCACCATGTTGGCCAGGCTGGTC
TTGAACTCCTGACCTCATGATCTGCCTGCCTTGGCCTCCCAAAGTGCTGGGATTACAGGC
ATGAGCCACCCTGCCCAGCCAATTTTCCATAACTTCTTTTTTTTTTTTTTGAGACAGAGT
CTCGCCCTGTCGCCCAGGCTGGAGTGCAGTGATGTGATCGTGGCTCACTGCAACCTCCAC
CTCCCGGGTTCAAGCGATTCTTCTGCCTCAGCCTCCCGAGTAGCCGGGACCACAGGTGCG
TGCCACCACGCCCGGCTAATCTTTGTAGTTTTTAGTAGAGACGGGGTTTTACCATGTTGG
CCAGGCAGATCTTGAACTTCTGACTTTGTGATCCACCCACCTCGGCCTCCCAAAGTGCTG
GGATTATAGGCATGAGCCACCGCACCCGGCCCTGGTTTTCCATAACTTCTAATGTGTAGA
GTTCCATTGGATTTTAAAAGAAATGATGCTTTTTTAAAAGCATCATTTTAAAAGCATTAA
AAGAAATGATACATGCTTATTATACAAAATCAAGTGATACATGATGGTACAGGAAAAAAT
ATTTAAAAATTCTACCACACACACACAACGTTTGGTGACATTTGGTGAACATCCTTTCAG
ATACTATGTGTATGTATTGGAAGACAAAAGAATGGCTAGAATAATTGGAAAAATGGGCTC
CTGCGACTTATTCCATTTTAGAAAAACTTAAGTCAATTAACACATTTTAATTGGCAAATA
AGGAACTAAAATGAATTCACTTTAGTGACATAGTTTTTCTAGAATTTAAAAACGTTCATC
ACATTCTTTTAAATTTTACATTTTTAAGAAGGATAAAATGGCCTGTAAATGCCAGGATTT
TTGGAATCGCAGGACTGGAAAGAAAAGCAACGCTGAGCCACTTGTTGCAGATTTTTGCTT
TTAGGGAGGACAACGGCAGAGTGCTGAGCTTGTTTTTGAAGTATTCTTAAAGAACGCGAG
GGATGAAGCCTCCAACTGCTTCCTCAGTAAATGGCCAACATTTACTGATCCTTTTGGTTC
ACAAGCATTTCACTTGCAATATGGTTTCAGGATTTCTTTTGGTATTTCTGTAGAGTGCAA
ATTGTTCAGAATCAAAATCATTCTGCTTTGCATGATTTTCTGGCTGAAACATTTGTAGTT
TTCACATTTGATTCATCTTTCTGATGAACTCAGTTGTCGTCTTGAAAGGGAGTGGGTACT
TTGTAAGACAGCTCAGCTGTTGAGTGAATGCATGGGGTTGCATGTGGGTGTGTTGGGGTC
AGAAGGGAGCCCTGTCAGAAGGTAGCTGTCATGAACTGGGTACACCGTCTGTTTGTGTGC
CTTTTCAAGACAACCATTCCATTAATGAAGCATTTTATAGGCACCCGTTCTTTATAGTAG
AGAAAGATGGCCAAAATGACTCTCAAATACATTTTTACTACACATGATATTTCAGCACAG
ATGAGTAGTAGGTCTTCAGGCAAAATTGCTATGCTATTATCCTATGAGTTGGAAGGTTAC
ATAAGAAGATTAGACATTTATAGAAATAAAACATGCCCATTAAAAGTGGGCAAAGGACAT
GAACAGACACTATATACAGATACTATTTGTAAGCGACTCAAGTTGGCTGTTGTTTTCTTC
CTCATTTGAAATACTGTATTGAAGACTTCATAACTCACATCATCTCTCTGTTGGCCCCTT
TTTCCTGGACAAATAGAAATATGAGAAAACGGTCTGAGGATCATGCTGTAACAAAGTGTT
TTATATCAGCTTTACTTGACCACTCATGCTTATGCCCTTGTAGCATCTTATCCATTGGGA
AAATGTTCCCAGCTGAAAACAGTCAATTGCAGCTGCAATTTTAAACATGGAATGCAATAG
ATAGACTTTAAGAAGTTAGTAAAATCTCTAAAACTATATGCAAAAATTTCTCTGCATGCT
TATATGCTTGCTTTTCTGAGGAATAGAAGCTTTTATTAACTGTTGTATTTAAAAATTTCA
ATTAGGCCGGGCTTGGTGGTTCATGCCTGTAATCCCAGCACTTTAGGGAGGCCGAAGCAG
GTGGATTGATCACCTAAGGGTATGAGTTCAAGACCAGCTTGGCCAACATGGTGAAATCCC
CGTCTCTACCAAAAATACAAAAAATTTGCTGGACATGGTGGCGTGCACCTGTAATCCCAG
CTACTCTGGAGGCTGAGGCAGGAGAATTGCTTGAGCCTAGGAGGCAGAGGTTGGAGTGAG
CTGAGATCACACCATTGCACTCCAGCCTGGGTGACAAGAGTGAATCTCCATCTCAAAAGA
AATTTTTTTAAATTAATTATTATTTGTTGAGACAGGGTCTCACTTTGTTGCCTGAGCTGT
GGGCAGTGGTGCAATCATGGTTCACTACAGCCTCAACCTCCTGGCTCAAGTGATCTTCCT
GCCTCCACCTCCCCAGTAGCTGGGACTTACAGGCGTGCATCACCACACCAGGCAAATTCT
TTTTTTAATGTTTTGTAGAGACGAGGCCTTGCTACGTTGCTTAGGCTGGTCTCAAACTCC
TGGGCTCATGCAATTCTCCTATCTTAGCATCTCGAAGAGGTAAGATAAAAGGCATGAGCC
ACTTCACCTGGCCTCATTAACTTTTCAAAGGAGTATATAAGTTTAAAAAGTCAGGATCCA
GTGATGTAAAACTTTAGTTTCCTTCCCTGTTGCCATTGAGCTCCTCCTTCTCCCTTTCTC
CTCCCCCTCCTTCTCCTTCTTCTCCTTCTTCCTCTTCCTCCTCCACCTCCTCCTCTTCTT
CTTCTCCTCTTCCTCCTGCTTCTCCTCTTCCTCCCCCCCTCCCCTCCTCCCCTTCCTCCT
CCCCTCCTCCCTCTCTTCCCCCTCCTCCCCGCCTCCTCCTCCCCCTCCTCCTCCCCCTCC
TCCTCCTCCTCCCCCTCCTCCTCCTCCCCCTCCTCCTCCTCTTTCTTCTTCTTTTTCTTT
CTCTTCTTCTTCCTCCTCCTCCCCAGTCGTCATCTTCTTCTTCCTTTTCTTCTTCTCCTT
CCTCCTCTTCTCCTTCTCCCTCCTCCTCCTTCTTCCTTCTTCCTTCTCTTCTTCTTCTTC
TTATCCTCCTCCTCCTTCATCTTCCTCCTCCTCCTCTTCTTCTCCTCCTCCTTATCCTTC
CTCCTCCCCCCTCCTCTTCTTCTTCTTCTTCTCCTTCTCCTTCTTCTTCCTCTTCTTCTT
CCTCCTCCTCCCCCCTCGTCGTCATCTTCTTCTTCTTCCTTTTCTTCTTCTCCTTTCTCT
TCTCCTTCTCCCTCCTCCTCCTTCTTCTTCCTTTTCTTCGTCTTCTTCTCCTTCCTCCTC
TTCTCCTTCTCCCTCCTCCTCCTTCTTCCTTCTTCCTTCTCTTCTTCTTCTTCTTCTCCT
CCTTCTCCTTCCTCCTCTTCCTCCTCCTCTTCCTCTTCCTCTTCTTCTTTCTTCCTTTTT
TACTTTGCCATAGAGACAATACTTTAGGTTCACTAAAATTTTTGCAGTTGTGGGATATTG
ATTCAAGCACCTGCTCATTTAAAGTGGTTGGCTATATTTAAAATGTTTTTATTGCTTTTT
ATCTCATACCCCCAAACTGTGCATTTCATCTCCAATTTCATTATTGGTTGAATTCCACAG
CCACAAATCATATGACTTAAAATCTTCTAACTTAGTTATTAATTTTATTGAAGATTTCCT
TTAGGGGTACTCTGGTTTTATAAATAGACTAATATGACACAATTCCTACTTAAAAACTAT
TTTAAATTTTTATTTTAATAGTTTTTGGGAAACAGGTGGTTTTCAATTACATTAATGAAT
TTTTTTGTTTGCAGATGATGCAATCTATTTTTTTAAATTATTATATTTTAAGTTCTGGGT
TACATATGCAGGTTTGTTACATAGGTAAACATGTGCCAGGTGGTTTGCTGCACCTATCAA
CCCATCACTTAGGTATTAAGCCCAGCATGCAATAGGTCTTTTCCCTACTGCTCTCCCCAC
CCGCCCTCCCCTGAAAGGCCTCTGTGTGTGTTGTTCCCCTCTCTGTGTCCAGATGTTCTC
ATGGTTCTGCTCCCACTTATAAGTGATAACATGTGGTGTTTGATTTTCTGTTCCTGTGTT
AGTTT
(chr17: 39,093,836-39,099,836):
Seq. ID. No. 2
CAAGGTAAAGGTTAGGAATGAATAAAGAAGCAGTCTGGGGGCGGGAGCCA
CGTGCTAGTTACTGGCAGTTGGATTTAAAGGGGACTTACAAATAAAATAT
CAGCGCAGGAAGGGCTTCTGGCCTTTCTGGTTCCAAGCCACTAATTGGAC
AGATGAGGAAAGCTGAGGCCTATGGTGGTGGGGAGGTTAGTGGCAGAGCC
AGGGCTAGATTCTTTCTCCACATAGCACTGTTATCTTGGGTTGAATCTTG
ACAGGTCCTGTACCACACTTCTCTTTCATAATAATAGGACTTGCCAATTG
GTGTAGCTCTTTTATATTCTTTATTGAATCCTCACAACTCTTTATGTATC
CAGGCAAGGGTTGTTGTCTTCATTTTGCAGACAAAGAATCAGGGTCTCAT
AGGGATGAATGATCCAGAGCTGATTCGCATGTACAGTGTAATTGAGCACA
TGAATTCATAGGTGACACTGCCCGAGTCTGTGCTGTCCTCCCTGCCTGGA
AAGTCACTCACCCCTACCTCTTCTATAGTTCAAGGGCTACCTCCTTCAAC
AATCATTTTCTGAATTCTCCTGCATGAGTTGGGTGACCCTCTAGGAGGAA
ACTGCCTCTTCTGGATGCCGAGGTCCTCCAGAGGGGCAGACACCTTGGCA
TCCCACCTCATGGTGGAAGCTGGGAGCCCCTAGCCAGGCGCAGCCACCTC
CTGTGCCTGGGATGAAGTCCACTCCGTTCTTTCACTCCATTATACTGCAT
TTGCCACACCCTTCTGAGACCGCAGGCTTCTTGAAAGCAGAGTTTATGTC
TTCCATTTTTGAGCACCAAAGTCTGGTTTCTTGGTGGTATTTTGCAAATG
TATGTTGACTCAATCATAAACTTTTTAGAGTCAGGATTTTAACACAGGTC
TCCTGATGCCATCAACTTCTGCTCCTTTTAGGACTGTGAGTTATAGAACT
AGAAAGTAGGAAAACGCCTCTCCTGGGTATAGATTAACCATCCATGAAGG
CCAATCTTTTCCAATTACAAATAAAGCCACCCAAAAGCACACCAGCTCTG
CTCCTCCCAGTCTTACAACAAAACCTTATCAGGGAAGAGTCTCTGAATAT
TTTTTATTTCACTGTGCACGAGCTGGAAAAATGAGCCTCCAAGCCTCAGG
GAAGCTTGGAGAGTGCTTAGAAAGAGTGCCGACAGTCACTTACATCTCAG
ATATGAGAAAAAGACAAAAGAGACAAGTGATAAAAATAGAGCAAATGAGG
GAACTAGAGAGACTTGTTTGGCGAGTGGTGGCAACCAGTGTTCTGTGGAT
GTGGACGGTTCCTGGTCATCAAAGTTATGGTCAGCTCTGGTTCCTGTTCG
CCTGGACCCAGACTTACCAAGGAAGAAAACATTTACCTTTAGCATGTGCA
AGGGCCGGGCTGCACTGCCTTTTTCTAAATTCTCTCACCAGAATCTACAA
TGAAATTAATGCTTCTCTAATTATTTCAGGCATGTCTGAGTGGCAGTGAC
TTATCAACCCCATAGATTTGGCCACTGTCTCATTTTATGTCCATGTGTGG
GAGTTTTGATTTATAGGACTTATCCTTGAGCAAAAGATTTCTTGTGTCAT
AGCAGGACCAGGGCCAGGAAAAACTGGTGGTGAGATCTGAGAGCACTGGG
GAGAAATAGTCTCTTCTAGTACTGTTATATGATGGTTTCCCATAACTTCT
TTTTTTTTTTTTCTCAAGATGGAGTTTTGCTCTGTCACCCAGGCTGGAGT
GCAATGGCGCAATCTTGACTCACTGCAACCTCCGCCTCCTGGGTTCAAGC
GATTCTCCTGCCTCAGCCTCCCGAGTAGCTAGGATTACAGGTGCGCACCA
CCATGCCCAGCTAATTTTTTGTATTTTTAGTAGAGACGGGGTTTCACCAT
GTTGGCCAGGCTGGTCTTGAACTCCTGACCTCATGATCTGCCTGCCTTGG
CCTCCCAAAGTGCTGGGATTACAGGCATGAGCCACCCTGCCCAGCCAATT
TTCCATAACTTCTTTTTTTTTTTTTTGAGACAGAGTCTCGCCCTGTCGCC
CAGGCTGGAGTGCAGTGATGTGATCGTGGCTCACTGCAACCTCCACCTCC
CGGGTTCAAGCGATTCTTCTGCCTCAGCCTCCCGAGTAGCCGGGACCACA
GGTGCGTGCCACCACGCCCGGCTAATCTTTGTAGTTTTTAGTAGAGACGG
GGTTTTACCATGTTGGCCAGGCAGATCTTGAACTTCTGACTTTGTGATCC
ACCCACCTCGGCCTCCCAAAGTGCTGGGATTATAGGCATGAGCCACCGCA
CCCGGCCCTGGTTTTCCATAACTTCTAATGTGTAGAGTTCCATTGGATTT
TAAAAGAAATGATGCTTTTTTAAAAGCATCATTTTAAAAGCATTAAAAGA
AATGATACATGCTTATTATACAAAATCAAGTGATACATGATGGTACAGGA
AAAAATATTTAAAAATTCTACCACACACACACAACGTTTGGTGACATTTG
GTGAACATCCTTTCAGATACTATGTGTATGTATTGGAAGACAAAAGAATG
GCTAGAATAATTGGAAAAATGGGCTCCTGCGACTTATTCCATTTTAGAAA
AACTTAAGTCAATTAACACATTTTAATTGGCAAATAAGGAACTAAAATGA
ATTCACTTTAGTGACATAGTTTTTCTAGAATTTAAAAACGTTCATCACAT
TCTTTTAAATTTTACATTTTTAAGAAGGATAAAATGGCCTGTAAATGCCA
GGATTTTTGGAATCGCAGGACTGGAAAGAAAAGCAACGCTGAGCCACTTG
TTGCAGATTTTTGCTTTTAGGGAGGACAACGGCAGAGTGCTGAGCTTGTT
TTTGAAGTATTCTTAAAGAACGCGAGGGATGAAGCCTCCAACTGCTTCCT
CAGTAAATGGCCAACATTTACTGATCCTTTTGGTTCACAAGCATTTCACT
TGCAATATGGTTTCAGGATTTCTTTTGGTATTTCTGTAGAGTGCAAATTG
TTCAGAATCAAAATCATTCTGCTTTGCATGATTTTCTGGCTGAAACATTT
GTAGTTTTCACATTTGATTCATCTTTCTGATGAACTCAGTTGTCGTCTTG
AAAGGGAGTGGGTACTTTGTAAGACAGCTCAGCTGTTGAGTGAATGCATG
GGGTTGCATGTGGGTGTGTTGGGGTCAGAAGGGAGCCCTGTCAGAAGGTA
GCTGTCATGAACTGGGTACACCGTCTGTTTGTGTGCCTTTTCAAGACAAC
CATTCCATTAATGAAGCATTTTATAGGCACCCGTTCTTTATAGTAGAGAA
AGATGGCCAAAATGACTCTCAAATACATTTTTACTACACATGATATTTCA
GCACAGATGAGTAGTAGGTCTTCAGGCAAAATTGCTATGCTATTATCCTA
TGAGTTGGAAGGTTACATAAGAAGATTAGACATTTATAGAAATAAAACAT
GCCCATTAAAAGTGGGCAAAGGACATGAACAGACACTATATACAGATACT
ATTTGTAAGCGACTCAAGTTGGCTGTTGTTTTCTTCCTCATTTGAAATAC
TGTATTGAAGACTTCATAACTCACATCATCTCTCTGTTGGCCCCTTTTTC
CTGGACAAATAGAAATATGAGAAAACGGTCTGAGGATCATGCTGTAACAA
AGTGTTTTATATCAGCTTTACTTGACCACTCATGCTTATGCCCTTGTAGC
ATCTTATCCATTGGGAAAATGTTCCCAGCTGAAAACAGTCAATTGCAGCT
GCAATTTTAAACATGGAATGCAATAGATAGACTTTAAGAAGTTAGTAAAA
TCTCTAAAACTATATGCAAAAATTTCTCTGCATGCTTATATGCTTGCTTT
TCTGAGGAATAGAAGCTTTTATTAACTGTTGTATTTAAAAATTTCAATTA
GGCCGGGCTTGGTGGTTCATGCCTGTAATCCCAGCACTTTAGGGAGGCCG
AAGCAGGTGGATTGATCACCTAAGGGTATGAGTTCAAGACCAGCTTGGCC
AACATGGTGAAATCCCCGTCTCTACCAAAAATACAAAAAATTTGCTGGAC
ATGGTGGCGTGCACCTGTAATCCCAGCTACTCTGGAGGCTGAGGCAGGAG
AATTGCTTGAGCCTAGGAGGCAGAGGTTGGAGTGAGCTGAGATCACACCA
TTGCACTCCAGCCTGGGTGACAAGAGTGAATCTCCATCTCAAAAGAAATT
TTTTTAAATTAATTATTATTTGTTGAGACAGGGTCTCACTTTGTTGCCTG
AGCTGTGGGCAGTGGTGCAATCATGGTTCACTACAGCCTCAACCTCCTGG
CTCAAGTGATCTTCCTGCCTCCACCTCCCCAGTAGCTGGGACTTACAGGC
GTGCATCACCACACCAGGCAAATTCTTTTTTTAATGTTTTGTAGAGACGA
GGCCTTGCTACGTTGCTTAGGCTGGTCTCAAACTCCTGGGCTCATGCAAT
TCTCCTATCTTAGCATCTCGAAGAGGTAAGATAAAAGGCATGAGCCACTT
CACCTGGCCTCATTAACTTTTCAAAGGAGTATATAAGTTTAAAAAGTCAG
GATCCAGTGATGTAAAACTTTAGTTTCCTTCCCTGTTGCCATTGAGCTCC
TCCTTCTCCCTTTCTCCTCCCCCTCCTTCTCCTTCTTCTCCTTCTTCCTC
TTCCTCCTCCACCTCCTCCTCTTCTTCTTCTCCTCTTCCTCCTGCTTCTC
CTCTTCCTCCCCCCCTCCCCTCCTCCCCTTCCTCCTCCCCTCCTCCCTCT
CTTCCCCCTCCTCCCCGCCTCCTCCTCCCCCTCCTCCTCCCCCTCCTCCT
CCTCCTCCCCCTCCTCCTCCTCCCCCTCCTCCTCCTCTTTCTTCTTCTTT
TTCTTTCTCTTCTTCTTCCTCCTCCTCCCCAGTCGTCATCTTCTTCTTCC
TTTTCTTCTTCTCCTTCCTCCTCTTCTCCTTCTCCCTCCTCCTCCTTCTT
CCTTCTTCCTTCTCTTCTTCTTCTTCTTATCCTCCTCCTCCTTCATCTTC
CTCCTCCTCCTCTTCTTCTCCTCCTCCTTATCCTTCCTCCTCCCCCCTCC
TCTTCTTCTTCTTCTTCTCCTTCTCCTTCTTCTTCCTCTTCTTCTTCCTC
CTCCTCCCCCCTCGTCGTCATCTTCTTCTTCTTCCTTTTCTTCTTCTCCT
TTCTCTTCTCCTTCTCCCTCCTCCTCCTTCTTCTTCCTTTTCTTCGTCTT
CTTCTCCTTCCTCCTCTTCTCCTTCTCCCTCCTCCTCCTTCTTCCTTCTT
CCTTCTCTTCTTCTTCTTCTTCTCCTCCTTCTCCTTCCTCCTCTTCCTCC
TCCTCTTCCTCTTCCTCTTCTTCTTTCTTCCTTTTTTACTTTGCCATAGA
GACAATACTTTAGGTTCACTAAAATTTTTGCAGTTGTGGGATATTGATTC
AAGCACCTGCTCATTTAAAGTGGTTGGCTATATTTAAAATGTTTTTATTG
CTTTTTATCTCATACCCCCAAACTGTGCATTTCATCTCCAATTTCATTAT
TGGTTGAATTCCACAGCCACAAATCATATGACTTAAAATCTTCTAACTTA
GTTATTAATTTTATTGAAGATTTCCTTTAGGGGTACTCTGGTTTTATAAA
TAGACTAATATGACACAATTCCTACTTAAAAACTATTTTAAATTTTTATT
TTAATAGTTTTTGGGAAACAGGTGGTTTTCAATTACATTAATGAATTTTT
TTGTTTGCAGATGATGCAATCTATTTTTTTAAATTATTATATTTTAAGTT
CTGGGTTACATATGCAGGTTTGTTACATAGGTAAACATGTGCCAGGTGGT
TTGCTGCACCTATCAACCCATCACTTAGGTATTAAGCCCAGCATGCAATA
GGTCTTTTCCCTACTGCTCTCCCCACCCGCCCTCCCCTGAAAGGCCTCTG
TGTGTGTTGTTCCCCTCTCTGTGTCCAGATGTTCTCATGGTTCTGCTCCC
ACTTATAAGTGATAACATGTGGTGTTTGATTTTCTGTTCCTGTGTTAGTT
T

1-13. (canceled)

14. A method of diagnosing and/or treating steatohepatitis in a patient comprising:
obtaining results of a determination of methylation of KRT23 DNA in a sample of human body fluid or human tissue;
diagnosing steatohepatitis if methylation of the KRT23 DNA is reduced compared to a sample of this human body fluid or tissue sample from a person with no steatohepatitis; and
treating the patient for steatohepatitis if it is diagnosed.

15. The method of claim 14, wherein the body fluid is blood or a blood derived sample.

16. The method of claim 15, wherein the body fluid is a serum or a plasma sample.

17. The method of claim 14, wherein the KRT23 DNA is free circulating DNA in a blood, plasma, or serum sample.

18. The method of claim 14, wherein methylation of KRT23 DNA is determined by bisulfite treatment.

19. The method of claim 14, wherein methylation of KRT23 DNA is determined by absolute quantitative analysis of methylated alleles (AQAMA).

20. The method of claim 14, wherein determination of methylation of the KRT23 DNA includes a determination of methylation status of at least 5 consecutive methylation sites.

21. The method of claim 20, wherein determination of methylation of the KRT23 DNA includes a determination of methylation status of at least 10 consecutive methylation sites.

22. The method of claim 21, wherein determination of methylation of the KRT23 DNA includes a determination of methylation status of at least 20 consecutive methylation sites.

23. The method of claim 22, wherein the determination of the methylation of KRT23 DNA includes a determination of methylation status of at least 30 consecutive methylation sites.

24. The method of claim 14, further defined as comprising using a kit for determining methylation status of KRT23 DNA in the sample, wherein the kit comprises at least one methylation determining agent for methylation of KRT23 DNA.

25. A diagnostic kit comprising a KRT23 binding primer pair which defines a methylation region in a KRT23 gene.

26. The kit of claim 25, wherein the primer pair is free of CpG motifs.

27. The kit of claim 25, wherein the primer pair is free of single nucleotide polymorphisms (SNPs).

28. The kit of claim 25, wherein the primer pair amplifies a region in KRT23 DNA that is at least 20 nucleotides long.

29. The kit of claim 28, wherein the primer pair amplifies a region in KRT23 DNA that is at least 50 nucleotides long.

30. The kit of claim 29, wherein the primer pair amplifies a region in KRT23 DNA that is at least 100 nucleotides long.

31. The kit of claim 25, wherein the primer pair amplifies a region in KRT23 DNA that contains at least 5 consecutive methylation sites of the KRT23 DNA.

32. The kit of claim 31, wherein the primer pair amplifies a region in KRT23 DNA that contains at least 10 consecutive methylation sites of the KRT23 DNA.

33. The kit of claim 32, wherein the primer pair amplifies a region in KRT23 DNA that contains at least 20 consecutive methylation sites of the KRT23 DNA.

34. The kit of claim 33, wherein the primer pair amplifies a region in KRT23 DNA that contains at least 30 consecutive methylation sites of KRT23 DNA.

35. The kit of claim 25, wherein the kit further comprises an agent containing bisulfite.

36. A method of diagnosing steatohepatitis in a patient comprising:
obtaining a diagnostic kit of claim 25; and
using the kit to make a determination of methylation of KRT23 DNA in a sample of human body fluid or human tissue from the patient;
wherein steatohepatitis is diagnosed in the patient if methylation of KRT23 DNA is reduced compared to a sample of this human body fluid or tissue sample from a person with no steatohepatitis.
